(19) United States
(12) Patent Application Publication
(10) Pub. No.: US 2014/0027750 A1
(43) Pub. Date: Jan. 30, 2014

(54) COMPOUND FOR ORGANIC OPTOELECTRONIC DEVICE, ORGANIC LIGHT EMITTING DIODE INCLUDING THE SAME AND DISPLAY INCLUDING THE ORGANIC LIGHT EMITTING DIODE

(71) Applicants: Eun-Sun YU, Uiwang-si (KR); Mi-Young CHAE, Uiwang-si (KR); Eui-Su KANG, Uiwang-si (KR); Nam-Heon LEE, Uiwang-si (KR)

(72) Inventors: Eun-Sun YU, Uiwang-si (KR); Mi-Young CHAE, Uiwang-si (KR); Eui-Su KANG, Uiwang-si (KR); Nam-Heon LEE, Uiwang-si (KR)

(21) Appl. No.: 14/042,981

(22) Filed: Oct. 1, 2013

Related U.S. Application Data
(63) Continuation of application No. PCT/KR2011/007315, filed on Oct. 4, 2011.

(30) Foreign Application Priority Data
May 26, 2011 (KR) 10-2011-0050344

(51) Int. Cl. H01L 51/00

(52) U.S. Cl. CPC H01L 51/0067; H01L 51/0072
USPC 257/40; 544/212; 546/276.7; 544/331

(57) ABSTRACT

A compound for an organic optoelectronic device, an organic light emitting diode including the same, and a display device including the organic light emitting diode, the compound being represented by the following Chemical Formula 1:

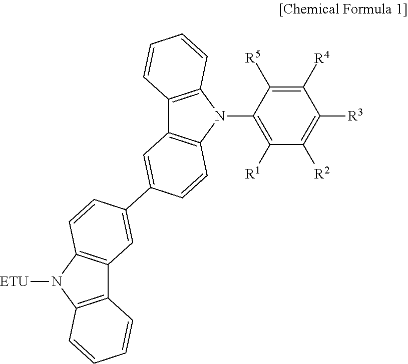

### CROSS-REFERENCE TO RELATED APPLICATION

Korean Patent Application No. 10-2011-0050344, filed on May, 26, 2011, in the Korean Intellectual Property Office, and entitled: “Compound For Organic Optoelectronic Device, Organic Light Emitting Diode Including the Same and Display Including the Organic Light Emitting Diode,” is incorporated by reference herein in its entirety.

This application is a continuation of pending International Application No. PCT/KR2011/007315, entitled “Compound For Organic Optoelectronic Device, Organic Light Emitting Diode Including the Same and Display Including the Organic Light Emitting Diode,” which was filed on Oct. 4, 2011, the entire contents of which are hereby incorporated by reference.

### 1. FIELD

Embodiments relate to a compound for an organic optoelectronic device, an organic light emitting diode including the same, and a display device including the organic light emitting diode.

### 2. DESCRIPTION OF THE RELATED ART

An organic optoelectric device is a device requiring a charge exchange between an electrode and an organic material by using a hole or an electron.

An organic optoelectric device may be classified as follows in accordance with its driving principles. One type of organic optoelectric device is an electron device driven as follows: excitons are generated in an organic material layer by photons from an external light source; the excitons are separated into electrons and holes; and the electrons and holes are transferred to different electrodes as a current source (voltage source).

Another type of organic optoelectric device is an electron device driven as follows: a voltage or a current is applied to at least two electrodes to inject holes and/or electrons into an organic material semiconductor positioned at an interface of the electrodes; and the device is driven by the injected electrons and holes.

The organic optoelectric device may include, e.g., an organic light emitting diode (OLED), an organic solar cell, an organic photo-conductor drum, an organic transistor, an organic memory device, or the like, and may include a hole injecting or transporting material, an electron injecting or transporting material, or a light emitting material.

The organic light emitting diode (OLED) may be particularly useful due to an increase in demand for flat panel displays. In general, organic light emission may refer to transformation of electrical energy to photo-energy.

The organic light emitting diode may transform electrical energy into light by applying current to an organic light emitting material. It may have a structure in which a functional organic material layer is interposed between an anode and a cathode. The organic material layer may include a multi-layer including different materials, e.g., a hole injection layer (HIL), a hole transport layer (HTL), an emission layer, an electron transport layer (ETL), and an electron injection layer (EIL), in order to help improve efficiency and stability of an organic light emitting diode.

In such an organic light emitting diode, when a voltage is applied between an anode and a cathode, holes from the anode and electrons from the cathode may be injected to an organic material layer. The generated excitons may generate light having certain wavelengths while shifting to a ground state.

### SUMMARY

Embodiments are directed to a compound for an organic optoelectronic device, an organic light emitting diode including the same, and a display device including the organic light emitting diode.

The embodiments may be realized by providing a compound for an organic optoelectronic device, the compound being represented by the following Chemical Formula 1:

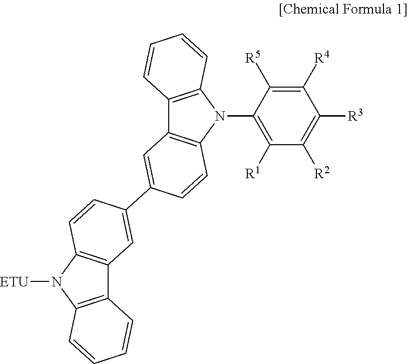

wherein, in Chemical Formula 1, ETU is a substituent having an electron property and is a substituted or unsubstituted C2 to C30 heteroaryl group, and R¹ to R⁵ are each independently hydrogen, deuterium, a substituted or unsubstituted C1 to C30 alkyl group, a substituted or unsubstituted C6 to C36 aryl group, or a combination thereof, provided that at least one of R¹ to R⁵ is a substituted or unsubstituted C1 to C30 alkyl group or a substituted or unsubstituted C6 to C36 aryl group.

The embodiments may also be realized by providing an organic light emitting diode including an anode, a cathode, and at least one organic thin layer between the anode and the cathode, wherein the at least one organic thin layer includes the compound for an organic optoelectronic device according to an embodiment.

The embodiments may also be realized by providing a display device including the organic light emitting diode according to an embodiment.

### BRIEF DESCRIPTION OF THE DRAWINGS

Features will be apparent to those of skill in the art by describing in detail exemplary embodiments with reference to the attached drawings in which:

FIGS. 1 to 5 illustrate cross-sectional views showing organic light emitting diodes including compounds according to various embodiments.

FIG. 6 illustrates a graph showing life-span data of organic light emitting diodes according to Examples 7, 9, and 11 and Comparative Example 4.

FIG. 7 illustrates a graph showing life-span data of organic light emitting diodes according to Examples 10 and 12 and Comparative Example 5.

FIG. 8 illustrates a graph showing life-span data of organic light emitting diodes according to Example 8 and Comparative Example 6.

### DETAILED DESCRIPTION

Exemplary embodiments will hereinafter be described in detail. However, these embodiments are only exemplary, and the embodiments are not limited thereto.

As used herein, when specific definition is not otherwise provided, the term “substituted” may refer to one substituted with deuterium, a halogen, a hydroxy group, an amino group, a substituted or unsubstituted C1 to C20 amine group, a nitro group, a substituted or unsubstituted C3 to C40 silyl group, a C1 to C30 alkyl group, a C1 to C10 alkylsilyl group, a C3 to C30 cyclo alkyl group, a C6 to C30 aryl group, a C1 to C20 alkoxy group, a fluoro group, a C1 to C10 trifluoro alkyl group such as a trifluoromethyl group, or a cyano group, instead of hydrogen.

Two adjacent substituents of the substituted a hydroxy group, amino group, a substituted or unsubstituted C1 to C20 amine group, nitro group, a substituted or unsubstituted C3 to C40 silyl group, a C1 to C30 alkyl group, a C1 to C10 an alkylsilyl group, C3 to C30 cycloalkyl group, a C6 to C30 aryl group, C1 to C20 alkoxy group, a C1 to C10 trifluoroalkyl group such as a trifluoromethyl group, or a cyano group may be linked to each other to provide a fused ring.

As used herein, when specific definition is not otherwise provided, the term “hetero” may refer to one including 1 to 3 of N, O, S, or P, and remaining carbons in one ring.

As used herein, when a definition is not otherwise provided, the term “combination thereof” may refer to at least two substituents bound to each other by a linker, or at least two substituents condensed to each other.

As used herein, when a definition is not otherwise provided, the term “alkyl group” may refer to an aliphatic hydrocarbon group. The alkyl may be a saturated alkyl group that does not include any double bond or triple bond.

Alternatively, the alkyl may be an unsaturated alkyl group that includes at least one double bond or triple bond.

The term “alkenylene group” may refer to a group in which at least two carbon atoms are bound in at least one carbon-carbon double bond, and the term “alkynylene group” may refer to a group in which at least two carbon atoms are bound in at least one carbon-carbon triple bond. Regardless of being saturated or unsaturated, the alkyl may be branched, linear, or cyclic.

The alkyl group may be a C1 to C20 alkyl group. For example, the alkyl group may be a C1 to C10 alkyl group or a C1 to C6 alkyl group.

For example, a C1 to C4 alkyl group may have 1 to 4 carbon atoms and may be selected from the group of methyl, ethyl, propyl, iso-propyl, n-butyl, iso-butyl, sec-butyl, and t-butyl.

Examples of the alkyl group may include a methyl group, an ethyl group, a propyl group, an isopropyl group, a butyl group, an isobutyl group, a t-butyl group, a pentyl group, a hexyl group, an ethenyl group, a propenyl group, a butenyl group, a cyclopropyl group, a cyclobutyl group, a cyclopentyl group, a cyclohexyl group, and the like.

The term “aromatic group” may refer to a cyclic functional group where all elements have conjugated p-orbital. Examples of the aromatic group may include an aryl group and a heteroaryl group.

The term “aryl group” may refer to an aryl group including a carbocyclic aryl (e.g., phenyl) having at least one ring having a covalent pi electron system.

The term “heteroaryl group” may refer to an aryl group where 1 to 3 heteroatoms selected from N, O, S, and P, and remaining carbon. When the heteroaryl group is a fused ring, each ring may include 1 to 3 heteroatoms.

As used herein, a carbazole-based derivative indicates a substituted or unsubstituted carbazolyl group including another hetero atom substituted for a nitrogen atom. For example, the substituted or unsubstituted carbazolyl group may include a dibenzofuranyl group, a dibenzothiophenyl group, and the like.

As used herein, hole properties may refer to properties in which holes generated at an anode are easily injected into an emission layer and moved therein due to conduction properties according to HOMO levels.

Electron properties refers to properties in which electrons generated at a cathode are easily injected into an emission layer and moved therein due to conduction properties according to LUMO levels.

According to an embodiment, a compound for an organic optoelectronic device may have a core including two carbazolyl groups and a phenyl group bonded with one of the two carbazolyl groups.

In an implementation, the phenyl group in the core may be bonded with at least one substituted or unsubstituted C1 to C30 alkyl group or substituted or unsubstituted C6 to C36 aryl group.

The core may further include a substituent having electron properties.

In an implementation, the core structure may include the substituent having electron properties combined with a carbazolyl group having hole properties. Thus, the compound may be applied to a light emitting material, a hole injection material, or a hole transport material for an organic optoelectronic device. For example, the compound may be applied to a light emitting material.

In an implementation, at least one substituted or unsubstituted C1 to C30 alkyl group or substituted or unsubstituted C6 to C36 aryl group bonded with the phenyl group in the core may help decrease molecular interaction. Thus, crystallinity of the compound may be lowered when the compound for an organic optoelectronic device is used to form a layer. As a result, recrystallization of the compound in a device may be suppressed.

As noted above, at least one substituent bonded to the core may have electron properties. Accordingly, the compound may be reinforced with electron properties as well as have a carbazole structure with excellent hole properties, which may satisfy conditions suitable for an emission layer. For example, the compound may be used as a host material for an emission layer.

In addition, the compound for an organic optoelectronic device may have various energy band gaps by introducing another substituent to the core moiety and a substituent substituted in the core moiety.

When the compound having an appropriate energy level (depending on a substituent) is used for an organic optoelectronic device, the compound may reinforce a hole transporting property or an electron transporting property of a layer. Thus, the organic optoelectronic device may have excellent efficiency and a driving voltage. In addition, the compound may have excellent electrochemical and thermal stability and thus, may help improve life-span characteristic of the organic optoelectronic device.

According to an embodiment, the compound for an organic optoelectronic device may be represented by the following Chemical Formula 1.

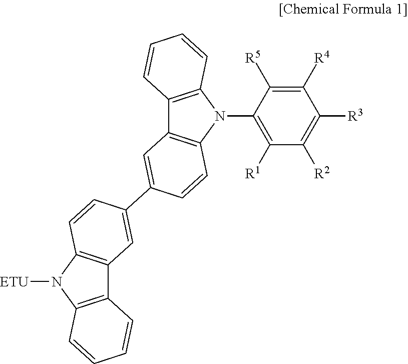

In Chemical Formula 1, ETU may be substituent having an electron property and may be a substituted or unsubstituted C2 to C30 heteroaryl group. R¹ to R⁵ may each independently be hydrogen, deuterium, a substituted or unsubstituted C1 to C30 alkyl group, a substituted or unsubstituted C6 to C36 aryl group, or a combination thereof. In an implementation, at least one of R¹ to R⁵ is a substituted or unsubstituted C1 to C30 alkyl group or a substituted or unsubstituted C6 to C36 aryl group.

In the compound for an organic optoelectronic device, at least one of R¹ to R⁵ may be a substituent selected from a substituted or unsubstituted C1 to C30 alkyl group and a substituted or unsubstituted C6 to C36 aryl group. The substituent may provide the compound for an organic optoelectronic device with light emitting, hole or electron properties; film stability; thermal stability, and high triplet excitation energy (T1).

The compound for an organic optoelectronic device including the substituent may not form a composite due to dipole-dipole strength among the molecules. If a composite were to be formed, HOMO/LUMO energy bandgap may be smaller than energy bandgap of a single molecule. If a compound that easily forms a composite were to be used for a device, the device may have undesirably decreased luminous efficiency and life-span.

The compound for an organic optoelectronic device including the substituent may not have a planar structure and may be less crystalline. A device fabricated by using a higher or highly crystalline compound easily may be degraded during the repetitive operation and thus, may exhibit undesirably decreased life-span.

The compound for an organic optoelectronic device including the substituent may have improved bulk characteristics. When the bulk characteristics of a compound are appropriately adjusted, a device may exhibit desired characteristics.

In an implementation, at least one of R² or R⁴ may be a substituted or unsubstituted C1 to C30 alkyl group or a substituted or unsubstituted C6 to C36 aryl group.

For example, at least one of R² or R⁴ may be a substituted or unsubstituted phenyl group. The substituted or unsubstituted phenyl group may include, e.g., a biphenyl group wherein the hydrogen of the phenyl group is substituted by an additional phenyl group. However, the substituted or unsubstituted phenyl group is not limited thereto.

In an implementation, at least one of R² to R⁴ may be a substituted or unsubstituted methyl group. However, R² to R⁴ are not limited thereto.

In an implementation, the ETU may include, e.g., a substituted or unsubstituted pyridinyl group, a substituted or unsubstituted pyrimidinyl group, a substituted or unsubstituted triazinyl group, or a combination thereof.

For example, the ETU may be a substituent represented by one of the following Chemical Formulas 2 to 6.

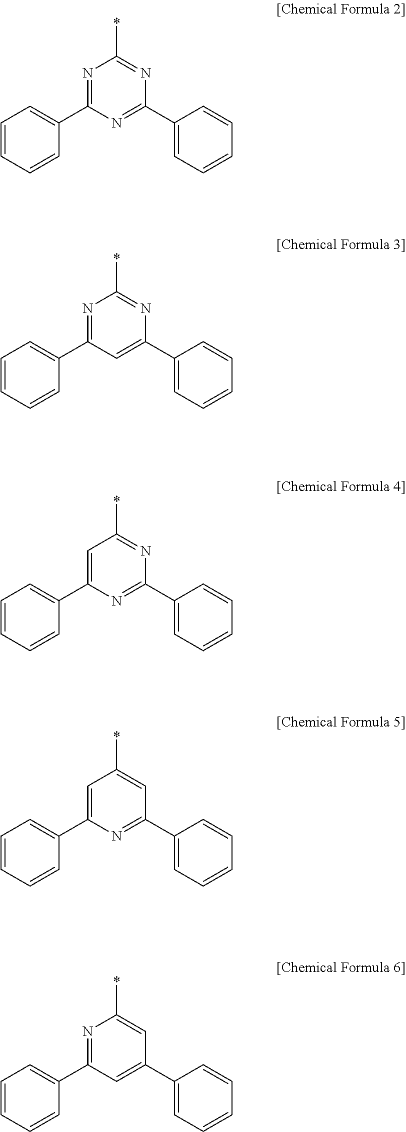 [SMILES: *c1nc(-c2ccccc2)nc(-c2ccccc2)n1 ; *c1nc(-c2ccccc2)cc(-c2ccccc2)n1 ; *c1cc(-c2ccccc2)nc(-c2ccccc2)n1 ; *c1cc(-c2ccccc2)nc(-c2ccccc2)c1 ; *c1cc(-c2ccccc2)cc(-c2ccccc2)n1]

In Chemical Formulae 2 to 6, * may represent a bonding location with a nitrogen atom in Chemical Formula 1, e.g., the nitrogen of one of the carbazole moieties.

In an implementation, the compound for an organic optoelectronic device may be represented by one of the following Chemical Formulae A-1 to A-39.

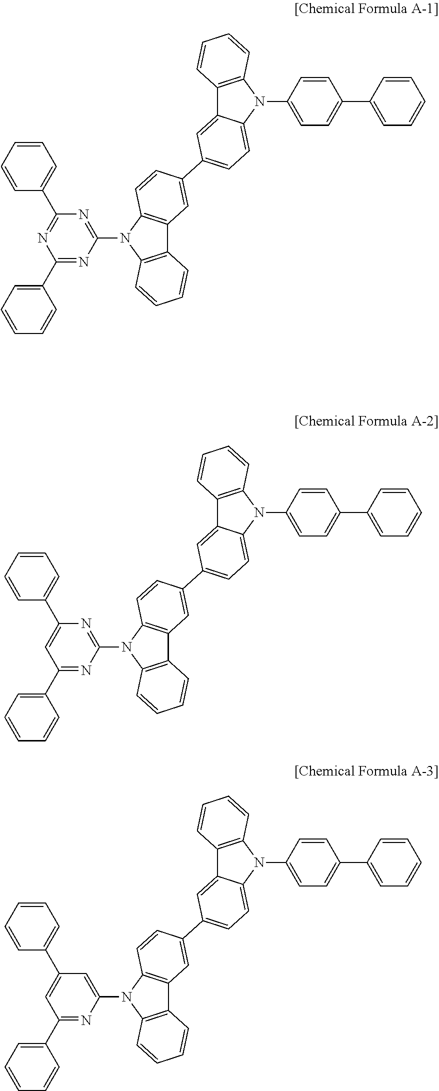 [SMILES: c1ccc(-c2ccc(-n3c4ccccc4c4cc(-c5ccc6c(c5)c5ccccc5n6-c5nc(-c6ccccc6)nc(-c6ccccc6)n5)ccc43)cc2)cc1 ; c1ccc(-c2ccc(-n3c4ccccc4c4cc(-c5ccc6c(c5)c5ccccc5n6-c5nc(-c6ccccc6)cc(-c6ccccc6)n5)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)n5)ccc43)cc2)cc1]

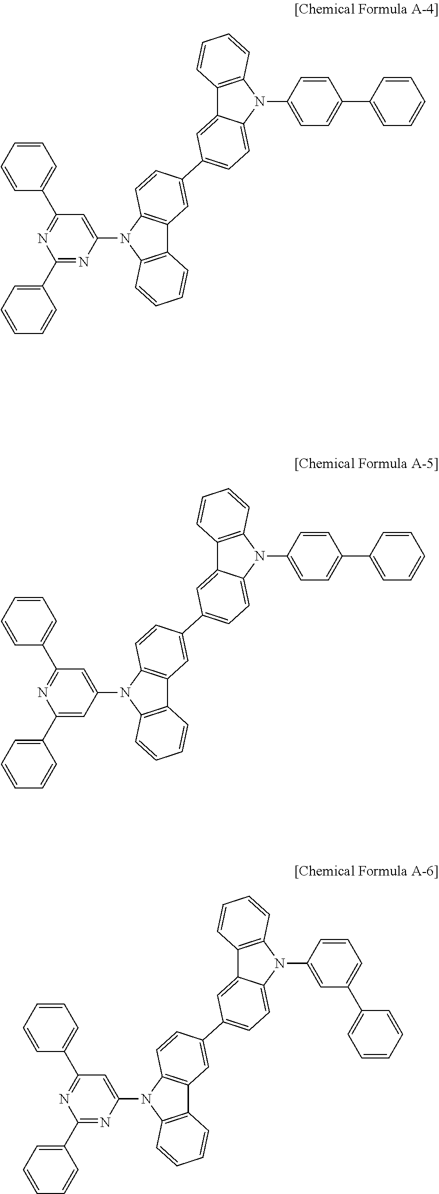 [SMILES: c1ccc(-c2ccc(-n3c4ccccc4c4cc(-c5ccc6c(c5)c5ccccc5n6-c5cc(-c6ccccc6)nc(-c6ccccc6)n5)ccc43)cc2)cc1 ; c1ccc(-c2ccc(-n3c4ccccc4c4cc(-c5ccc6c(c5)c5ccccc5n6-c5cc(-c6ccccc6)nc(-c6ccccc6)c5)ccc43)cc2)cc1 ; c1ccc(-c2cccc(-n3c4ccccc4c4cc(-c5ccc6c(c5)c5ccccc5n6-c5cc(-c6ccccc6)nc(-c6ccccc6)n5)ccc43)c2)cc1]

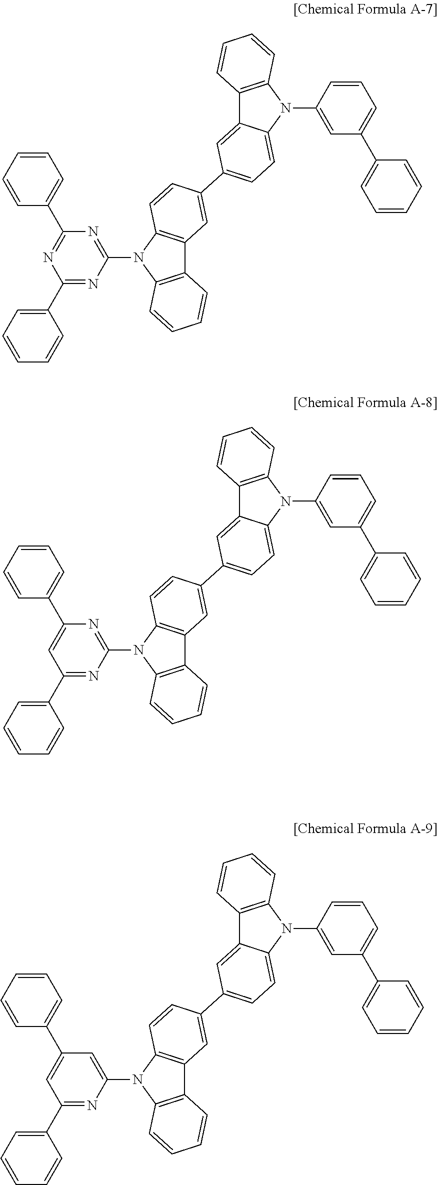 [SMILES: c1ccc(-c2cccc(-n3c4ccccc4c4cc(-c5ccc6c(c5)c5ccccc5n6-c5nc(-c6ccccc6)nc(-c6ccccc6)n5)ccc43)c2)cc1 ; c1ccc(-c2cccc(-n3c4ccccc4c4cc(-c5ccc6c(c5)c5ccccc5n6-c5nc(-c6ccccc6)cc(-c6ccccc6)n5)ccc43)c2)cc1 ; c1ccc(-c2cccc(-n3c4ccccc4c4cc(-c5ccc6c(c5)c5ccccc5n6-c5cc(-c6ccccc6)cc(-c6ccccc6)n5)ccc43)c2)cc1]

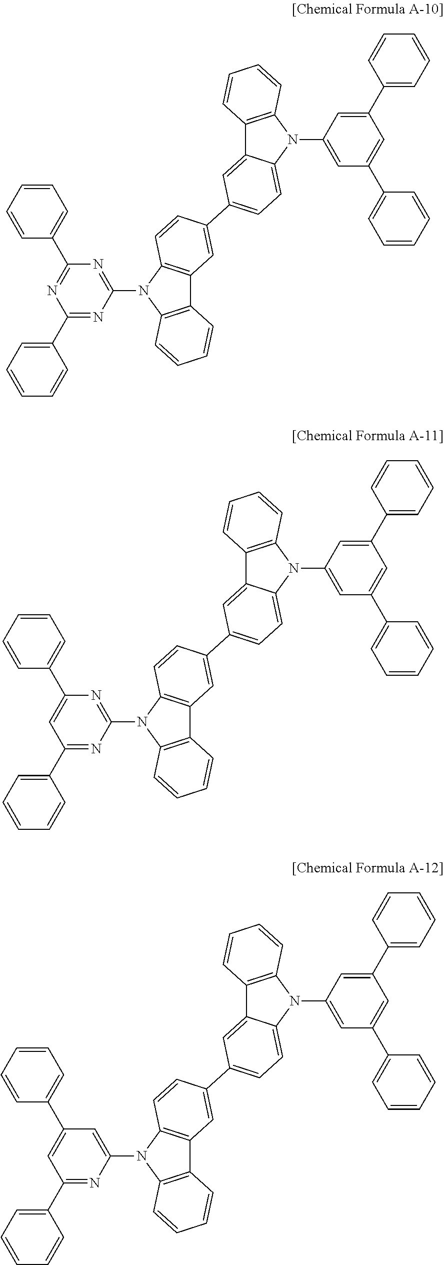 [SMILES: c1ccc(-c2cc(-c3ccccc3)cc(-n3c4ccccc4c4cc(-c5ccc6c(c5)c5ccccc5n6-c5nc(-c6ccccc6)nc(-c6ccccc6)n5)ccc43)c2)cc1 ; c1ccc(-c2cc(-c3ccccc3)cc(-n3c4ccccc4c4cc(-c5ccc6c(c5)c5ccccc5n6-c5nc(-c6ccccc6)cc(-c6ccccc6)n5)ccc43)c2)cc1 ; c1ccc(-c2cc(-c3ccccc3)cc(-n3c4ccccc4c4cc(-c5ccc6c(c5)c5ccccc5n6-c5cc(-c6ccccc6)cc(-c6ccccc6)n5)ccc43)c2)cc1]

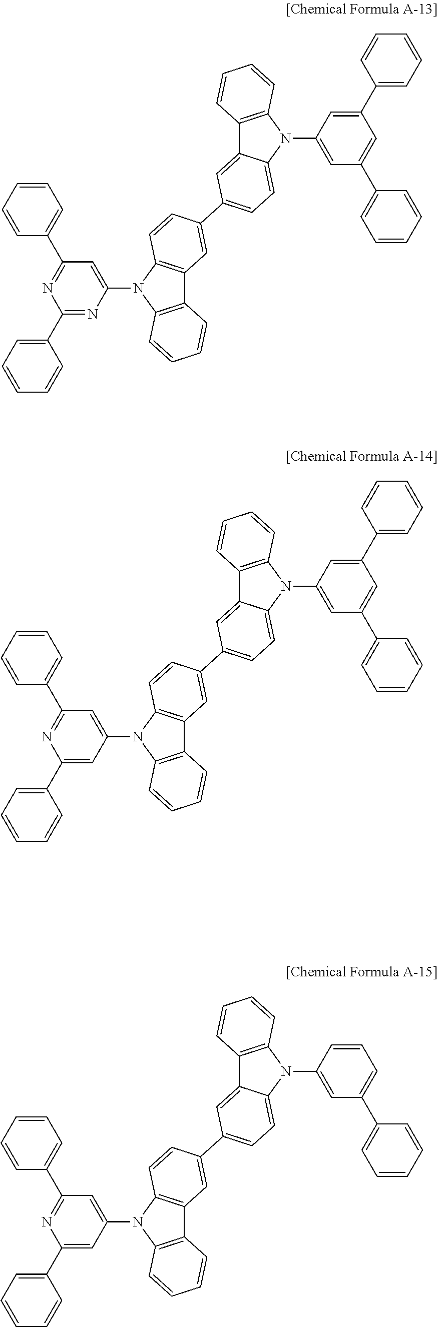 [SMILES: c1ccc(-c2cc(-c3ccccc3)cc(-n3c4ccccc4c4cc(-c5ccc6c(c5)c5ccccc5n6-c5cc(-c6ccccc6)nc(-c6ccccc6)n5)ccc43)c2)cc1 ; c1ccc(-c2cc(-c3ccccc3)cc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cccc(-n3c4ccccc4c4cc(-c5ccc6c(c5)c5ccccc5n6-c5cc(-c6ccccc6)nc(-c6ccccc6)c5)ccc43)c2)cc1]

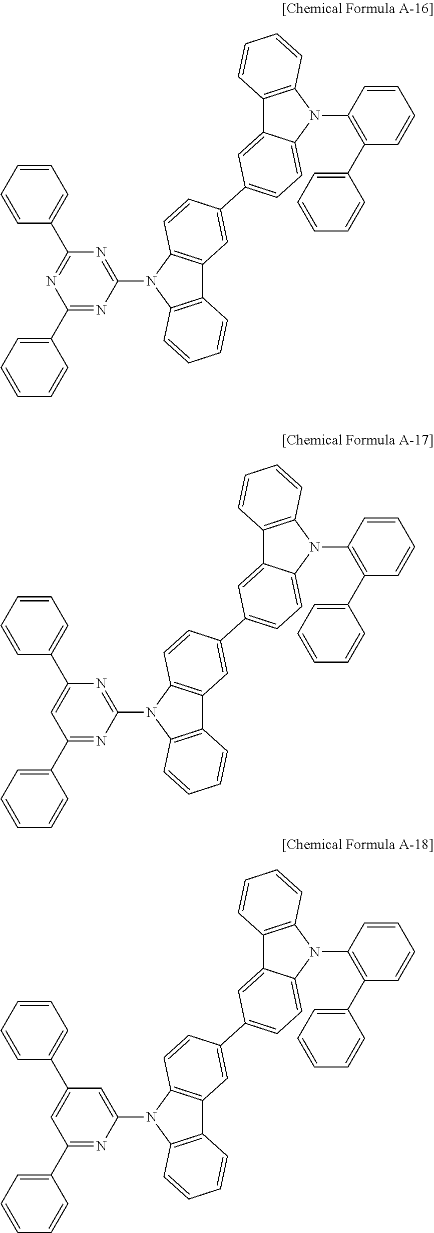 [SMILES: c1ccc(-c2nc(-c3ccccc3)nc(-n3c4ccccc4c4cc(-c5ccc6c(c5)c5ccccc5n6-c5ccccc5-c5ccccc5)ccc43)n2)cc1 ; c1ccc(-c2cc(-c3ccccc3)nc(-n3c4ccccc4c4cc(-c5ccc6c(c5)c5ccccc5n6-c5ccccc5-c5ccccc5)ccc43)n2)cc1 ; c1ccc(-c2cc(-c3ccccc3)nc(-n3c4ccccc4c4cc(-c5ccc6c(c5)c5ccccc5n6-c5ccccc5-c5ccccc5)ccc43)c2)cc1]

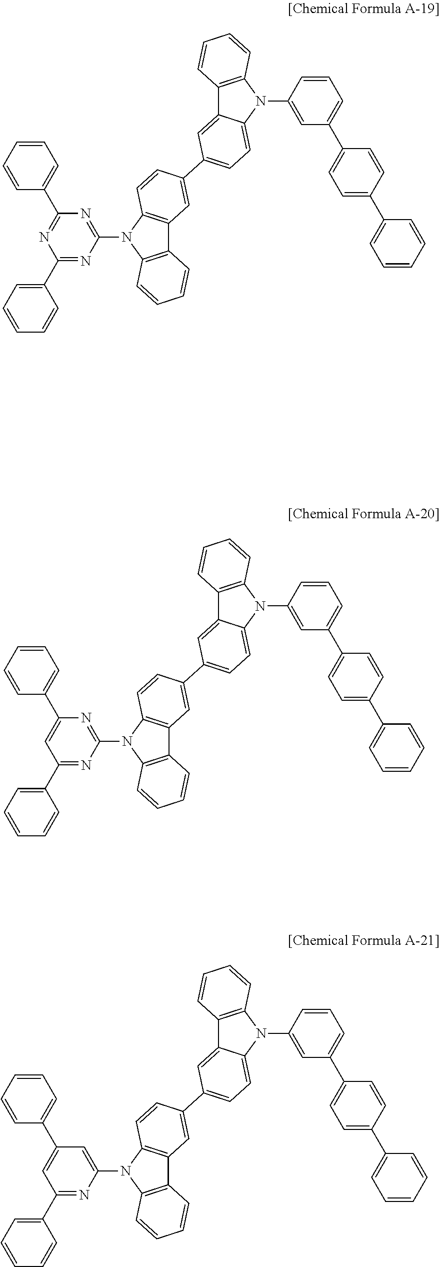 [SMILES: c1ccc(-c2ccc(-c3cccc(-n4c5ccccc5c5cc(-c6ccc7c(c6)c6ccccc6n7-c6nc(-c7ccccc7)nc(-c7ccccc7)n6)ccc54)c3)cc2)cc1 ; c1ccc(-c2ccc(-c3cccc(-n4c5ccccc5c5cc(-c6ccc7c(c6)c6ccccc6n7-c6nc(-c7ccccc7)cc(-c7ccccc7)n6)ccc54)c3)cc2)cc1 ; c1ccc(-c2ccc(-c3cccc(-n4c5ccccc5c5cc(-c6ccc7c(c6)c6ccccc6n7-c6cc(-c7ccccc7)cc(-c7ccccc7)n6)ccc54)c3)cc2)cc1]

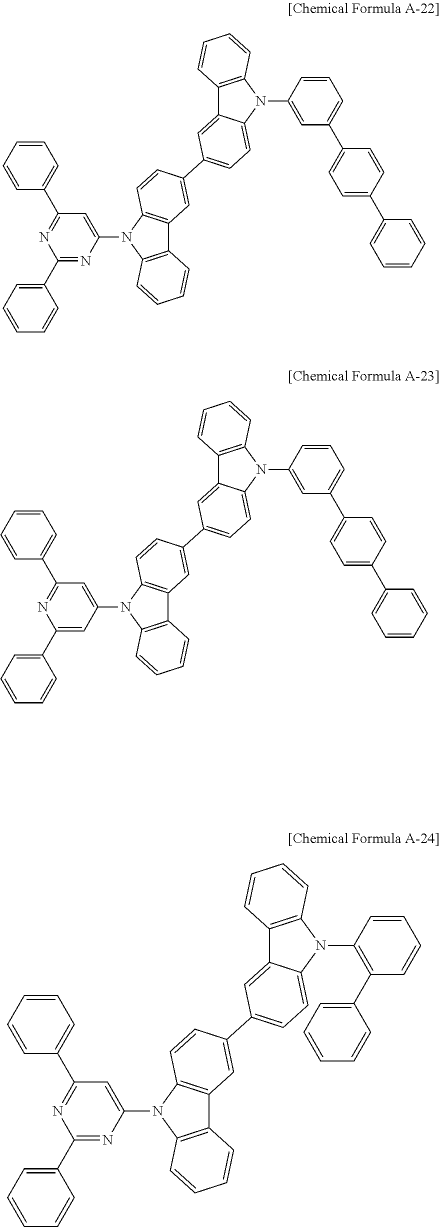 [SMILES: c1ccc(-c2ccc(-c3cccc(-n4c5ccccc5c5cc(-c6ccc7c(c6)c6ccccc6n7-c6cc(-c7ccccc7)nc(-c7ccccc7)n6)ccc54)c3)cc2)cc1 ; c1ccc(-c2ccc(-c3cccc(-n4c5ccccc5c5cc(-c6ccc7c(c6)c6ccccc6n7-c6cc(-c7ccccc7)nc(-c7ccccc7)c6)ccc54)c3)cc2)cc1 ; c1ccc(-c2cc(-n3c4ccccc4c4cc(-c5ccc6c(c5)c5ccccc5n6-c5ccccc5-c5ccccc5)ccc43)nc(-c3ccccc3)n2)cc1]

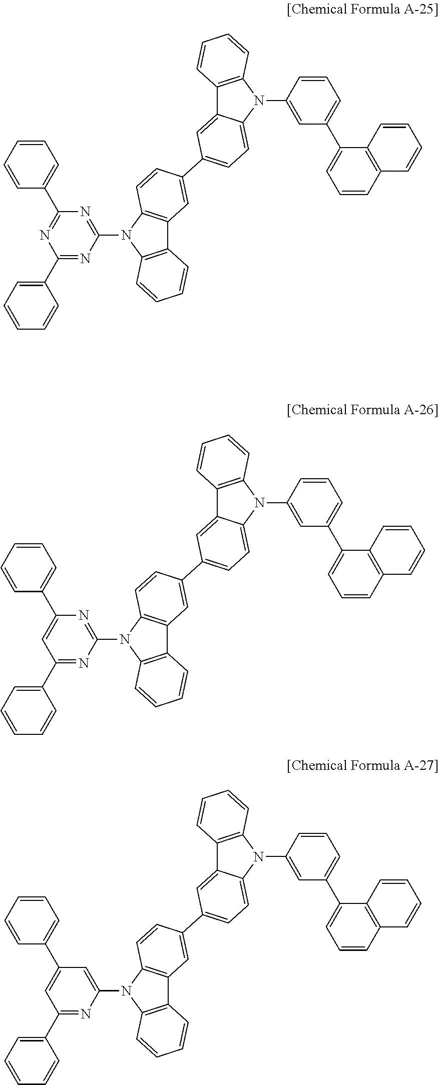 [SMILES: c1ccc(-c2nc(-c3ccccc3)nc(-n3c4ccccc4c4cc(-c5ccc6c(c5)c5ccccc5n6-c5cccc(-c6cccc7ccccc67)c5)ccc43)n2)cc1 ; c1ccc(-c2cc(-c3ccccc3)nc(-n3c4ccccc4c4cc(-c5ccc6c(c5)c5ccccc5n6-c5cccc(-c6cccc7ccccc67)c5)ccc43)n2)cc1 ; c1ccc(-c2cc(-c3ccccc3)nc(-n3c4ccccc4c4cc(-c5ccc6c(c5)c5ccccc5n6-c5cccc(-c6cccc7ccccc67)c5)ccc43)c2)cc1]

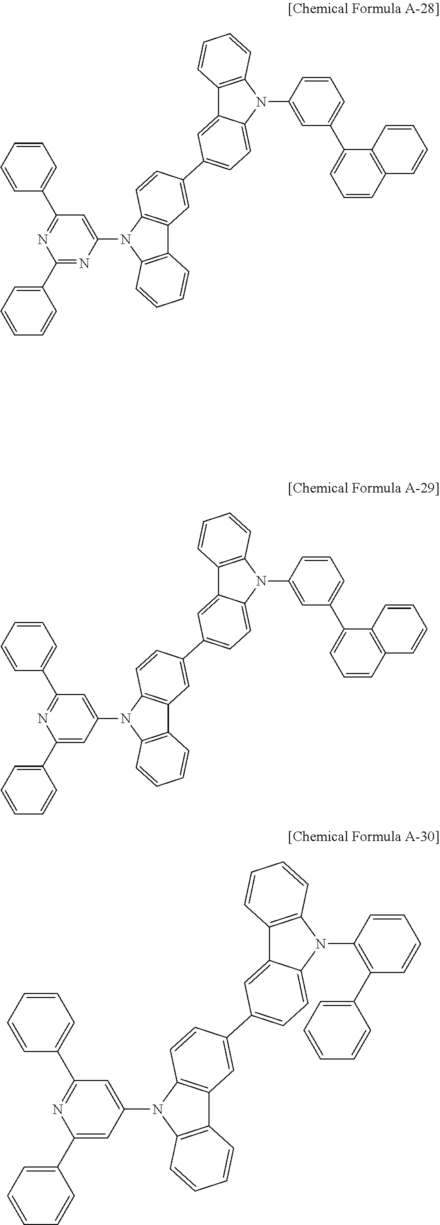 [SMILES: c1ccc(-c2cc(-n3c4ccccc4c4cc(-c5ccc6c(c5)c5ccccc5n6-c5cccc(-c6cccc7ccccc67)c5)ccc43)nc(-c3ccccc3)n2)cc1 ; c1ccc(-c2cc(-n3c4ccccc4c4cc(-c5ccc6c(c5)c5ccccc5n6-c5cccc(-c6cccc7ccccc67)c5)ccc43)cc(-c3ccccc3)n2)cc1 ; c1ccc(-c2cc(-n3c4ccccc4c4cc(-c5ccc6c(c5)c5ccccc5n6-c5ccccc5-c5ccccc5)ccc43)cc(-c3ccccc3)n2)cc1]

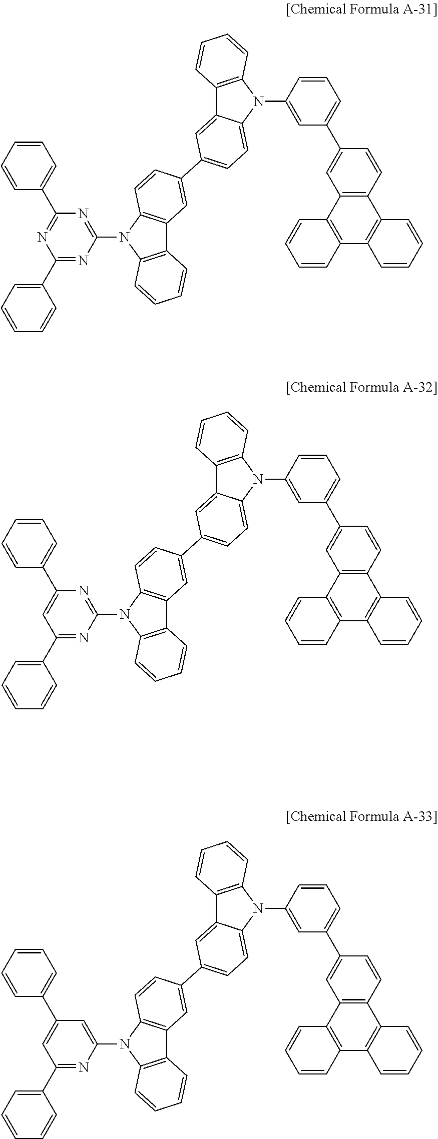 [SMILES: c1ccc(-c2nc(-c3ccccc3)nc(-n3c4ccccc4c4cc(-c5ccc6c(c5)c5ccccc5n6-c5cccc(-c6ccc7c8ccccc8c8ccccc8c7c6)c5)ccc43)n2)cc1 ; c1ccc(-c2cc(-c3ccccc3)nc(-n3c4ccccc4c4cc(-c5ccc6c(c5)c5ccccc5n6-c5cccc(-c6ccc7c8ccccc8c8ccccc8c7c6)c5)ccc43)n2)cc1 ; c1ccc(-c2cc(-c3ccccc3)nc(-n3c4ccccc4c4cc(-c5ccc6c(c5)c5ccccc5n6-c5cccc(-c6ccc7c8ccccc8c8ccccc8c7c6)c5)ccc43)c2)cc1]

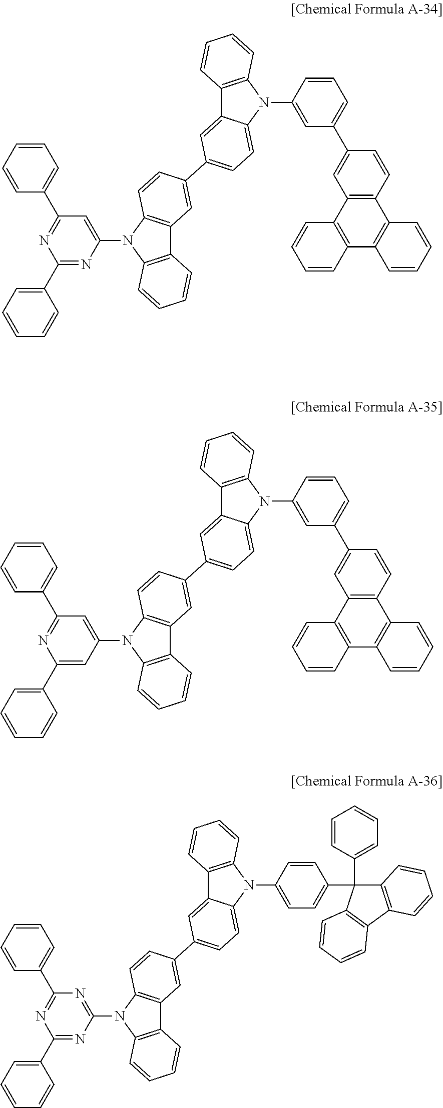 [SMILES: c1ccc(-c2cc(-n3c4ccccc4c4cc(-c5ccc6c(c5)c5ccccc5n6-c5cccc(-c6ccc7c8ccccc8c8ccccc8c7c6)c5)ccc43)nc(-c3ccccc3)n2)cc1 ; c1ccc(-c2cc(-n3c4ccccc4c4cc(-c5ccc6c(c5)c5ccccc5n6-c5cccc(-c6ccc7c8ccccc8c8ccccc8c7c6)c5)ccc43)cc(-c3ccccc3)n2)cc1 ; c1ccc(-c2nc(-c3ccccc3)nc(-n3c4ccccc4c4cc(-c5ccc6c(c5)c5ccccc5n6-c5ccc(C6(c7ccccc7)c7ccccc7-c7ccccc76)cc5)ccc43)n2)cc1]

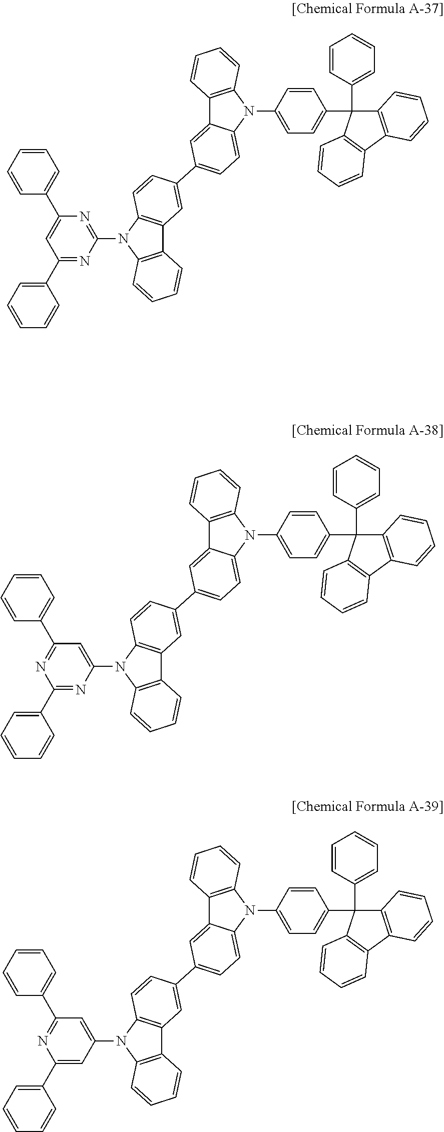 [SMILES: c1ccc(-c2cc(-c3ccccc3)nc(-n3c4ccccc4c4cc(-c5ccc6c(c5)c5ccccc5n6-c5ccc(C6(c7ccccc7)c7ccccc7-c7ccccc76)cc5)ccc43)n2)cc1 ; c1ccc(-c2cc(-n3c4ccccc4c4cc(-c5ccc6c(c5)c5ccccc5n6-c5ccc(C6(c7ccccc7)c7ccccc7-c7ccccc76)cc5)ccc43)nc(-c3ccccc3)n2)cc1 ; c1ccc(-c2cc(-n3c4ccccc4c4cc(-c5ccc6c(c5)c5ccccc5n6-c5ccc(C6(c7ccccc7)c7ccccc7-c7ccccc76)cc5)ccc43)cc(-c3ccccc3)n2)cc1]

In an implementation, the compound for an organic optoelectronic device may be represented by one of the following Chemical Formulae B-1 to B-25.

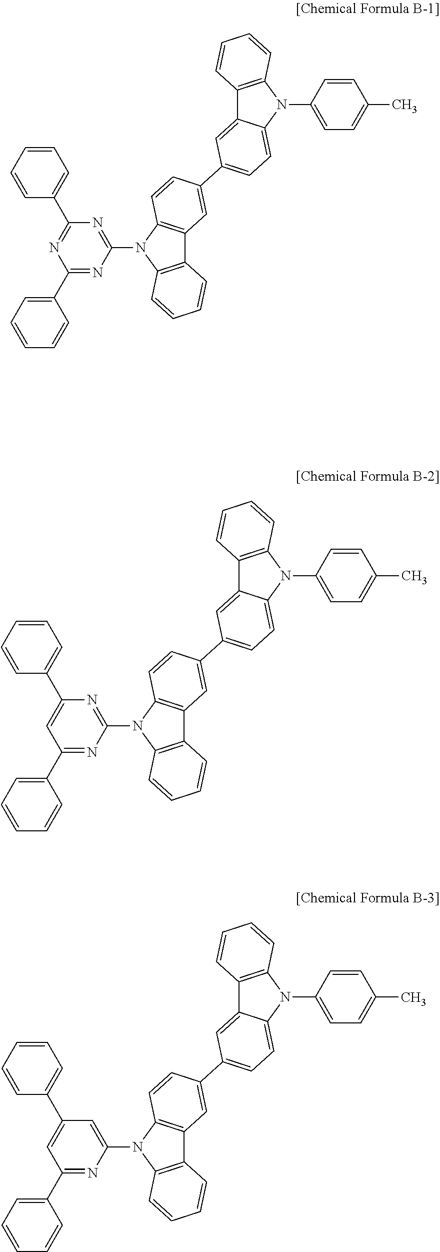 [SMILES: Cc1ccc(-n2c3ccccc3c3cc(-c4ccc5c(c4)c4ccccc4n5-c4nc(-c5ccccc5)nc(-c5ccccc5)n4)ccc32)cc1 ; Cc1ccc(-n2c3ccccc3c3cc(-c4ccc5c(c4)c4ccccc4n5-c4nc(-c5ccccc5)cc(-c5ccccc5)n4)ccc32)cc1 ; Cc1ccc(-n2c3ccccc3c3cc(-c4ccc5c(c4)c4ccccc4n5-c4cc(-c5ccccc5)cc(-c5ccccc5)n4)ccc32)cc1]

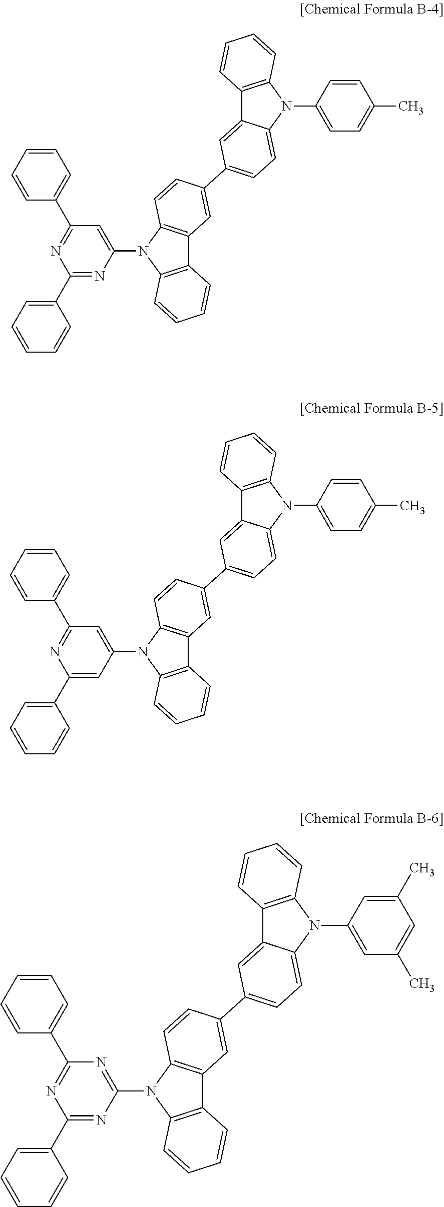 [SMILES: Cc1ccc(-n2c3ccccc3c3cc(-c4ccc5c(c4)c4ccccc4n5-c4cc(-c5ccccc5)nc(-c5ccccc5)n4)ccc32)cc1 ; Cc1ccc(-n2c3ccccc3c3cc(-c4ccc5c(c4)c4ccccc4n5-c4cc(-c5ccccc5)nc(-c5ccccc5)c4)ccc32)cc1 ; Cc1cc(C)cc(-n2c3ccccc3c3cc(-c4ccc5c(c4)c4ccccc4n5-c4nc(-c5ccccc5)nc(-c5ccccc5)n4)ccc32)c1]

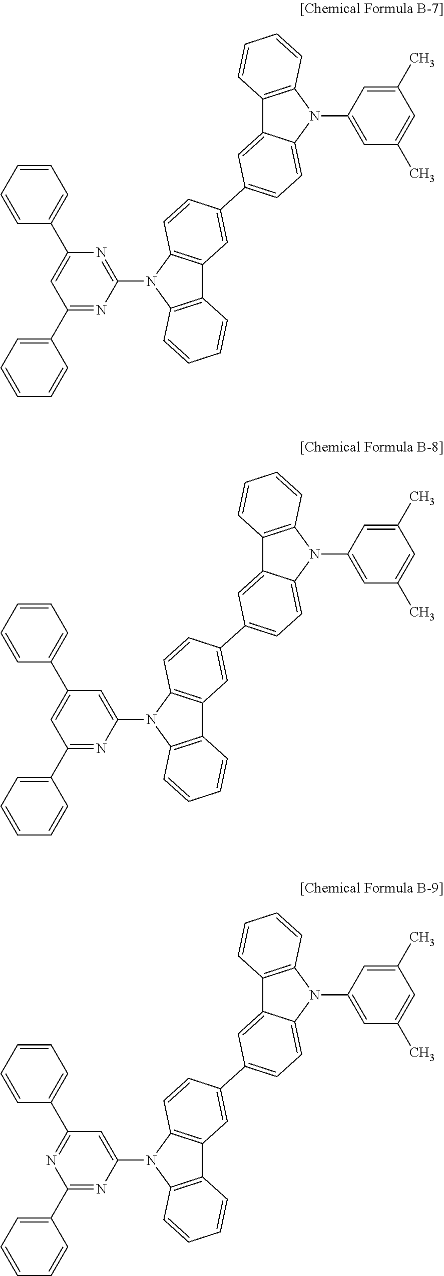 [SMILES: Cc1cc(C)cc(-n2c3ccccc3c3cc(-c4ccc5c(c4)c4ccccc4n5-c4nc(-c5ccccc5)cc(-c5ccccc5)n4)ccc32)c1 ; Cc1cc(C)cc(-n2c3ccccc3c3cc(-c4ccc5c(c4)c4ccccc4n5-c4cc(-c5ccccc5)cc(-c5ccccc5)n4)ccc32)c1 ; Cc1cc(C)cc(-n2c3ccccc3c3cc(-c4ccc5c(c4)c4ccccc4n5-c4cc(-c5ccccc5)nc(-c5ccccc5)n4)ccc32)c1]

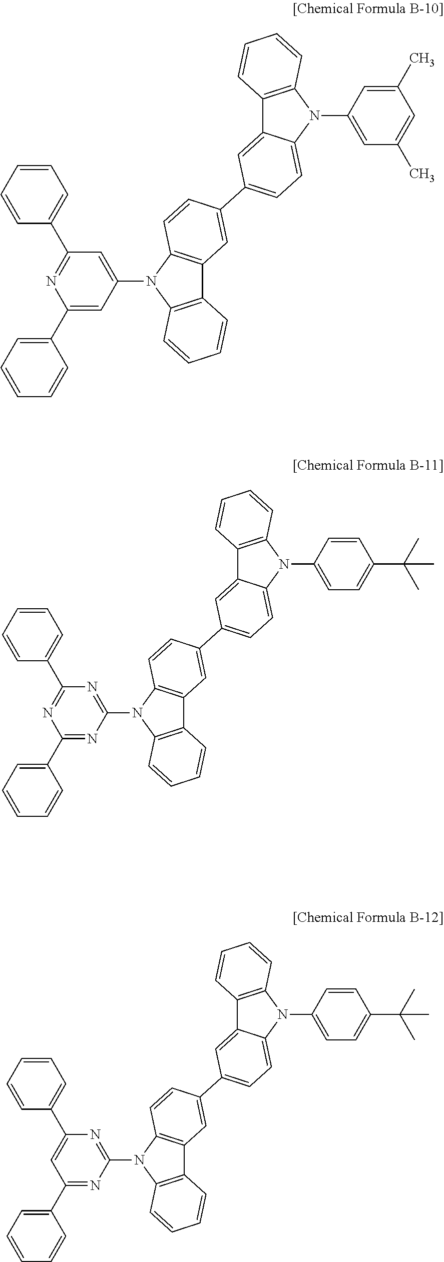 [SMILES: Cc1cc(C)cc(-n2c3ccccc3c3cc(-c4ccc5c(c4)c4ccccc4n5-c4cc(-c5ccccc5)nc(-c5ccccc5)c4)ccc32)c1 ; CC(C)(C)c1ccc(-n2c3ccccc3c3cc(-c4ccc5c(c4)c4ccccc4n5-c4nc(-c5ccccc5)nc(-c5ccccc5)n4)ccc32)cc1 ; CC(C)(C)c1ccc(-n2c3ccccc3c3cc(-c4ccc5c(c4)c4ccccc4n5-c4nc(-c5ccccc5)cc(-c5ccccc5)n4)ccc32)cc1]

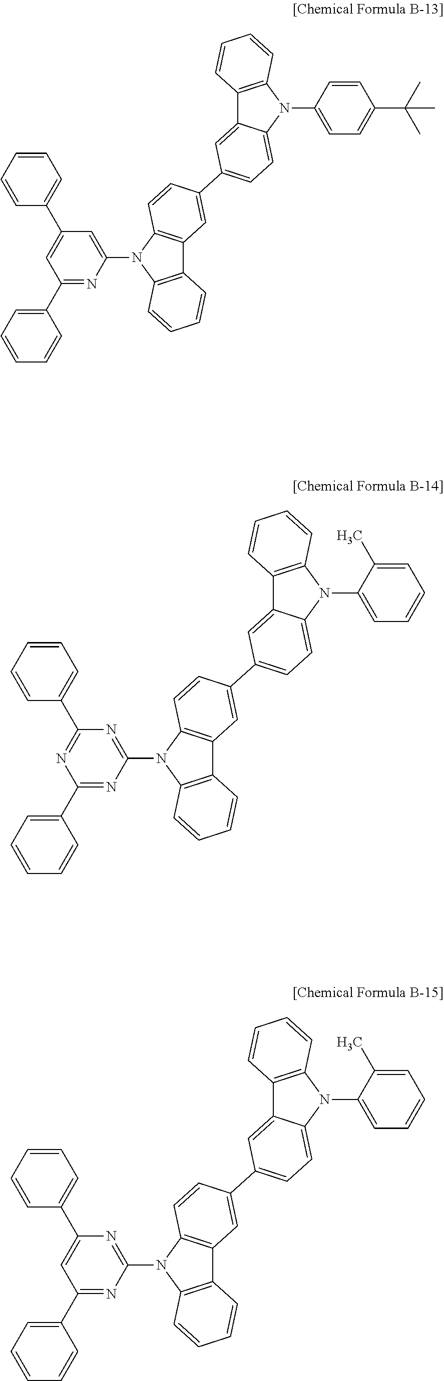 [SMILES: CC(C)(C)c1ccc(-n2c3ccccc3c3cc(-c4ccc5c(c4)c4ccccc4n5-c4cc(-c5ccccc5)cc(-c5ccccc5)n4)ccc32)cc1 ; Cc1ccccc1-n1c2ccccc2c2cc(-c3ccc4c(c3)c3ccccc3n4-c3nc(-c4ccccc4)nc(-c4ccccc4)n3)ccc21 ; Cc1ccccc1-n1c2ccccc2c2cc(-c3ccc4c(c3)c3ccccc3n4-c3nc(-c4ccccc4)cc(-c4ccccc4)n3)ccc21]

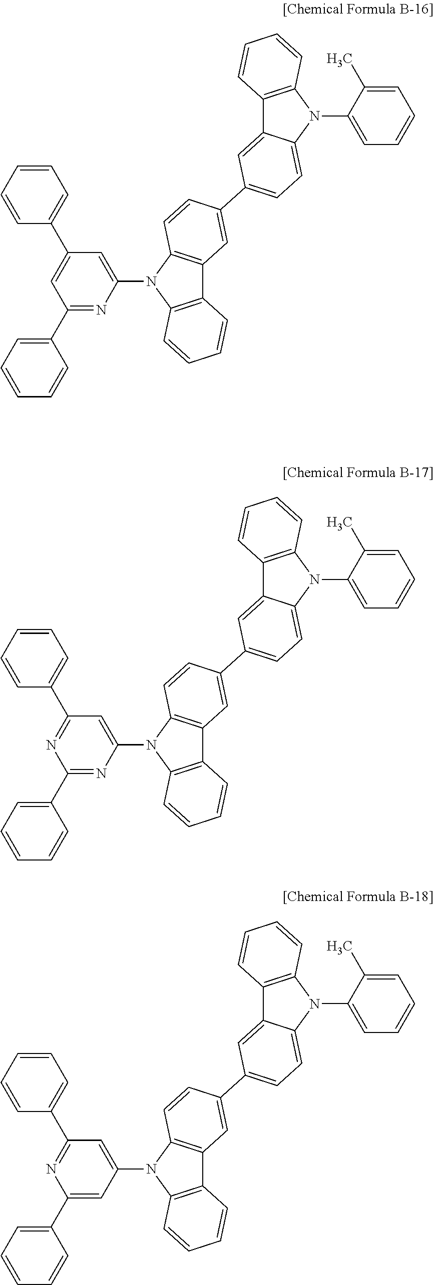 [SMILES: Cc1ccccc1-n1c2ccccc2c2cc(-c3ccc4c(c3)c3ccccc3n4-c3cc(-c4ccccc4)cc(-c4ccccc4)n3)ccc21 ; Cc1ccccc1-n1c2ccccc2c2cc(-c3ccc4c(c3)c3ccccc3n4-c3cc(-c4ccccc4)nc(-c4ccccc4)n3)ccc21 ; Cc1ccccc1-n1c2ccccc2c2cc(-c3ccc4c(c3)c3ccccc3n4-c3cc(-c4ccccc4)nc(-c4ccccc4)c3)ccc21]

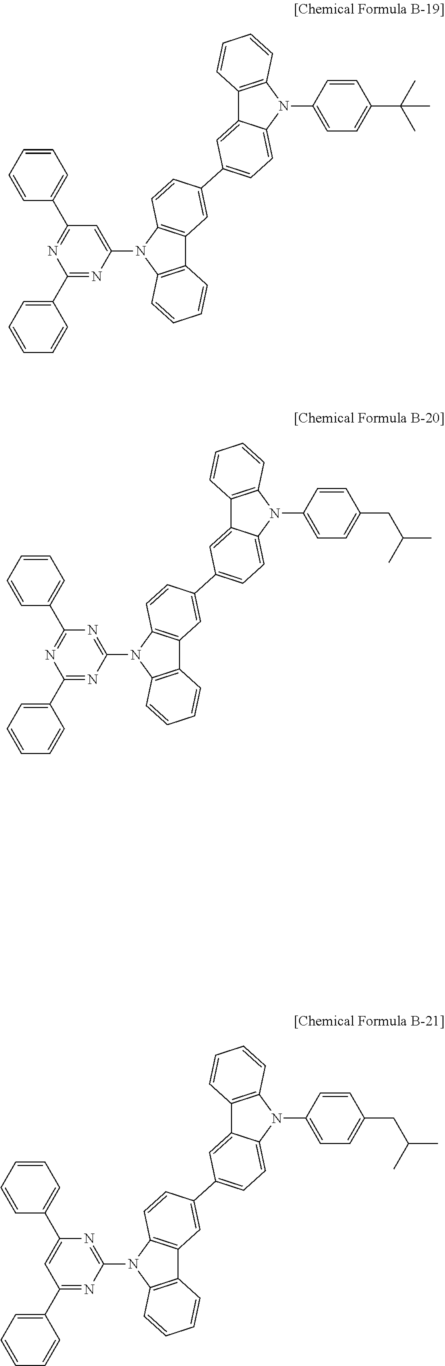 [SMILES: CC(C)(C)c1ccc(-n2c3ccccc3c3cc(-c4ccc5c(c4)c4ccccc4n5-c4cc(-c5ccccc5)nc(-c5ccccc5)n4)ccc32)cc1 ; CC(C)Cc1ccc(-n2c3ccccc3c3cc(-c4ccc5c(c4)c4ccccc4n5-c4nc(-c5ccccc5)nc(-c5ccccc5)n4)ccc32)cc1 ; CC(C)Cc1ccc(-n2c3ccccc3c3cc(-c4ccc5c(c4)c4ccccc4n5-c4nc(-c5ccccc5)cc(-c5ccccc5)n4)ccc32)cc1]

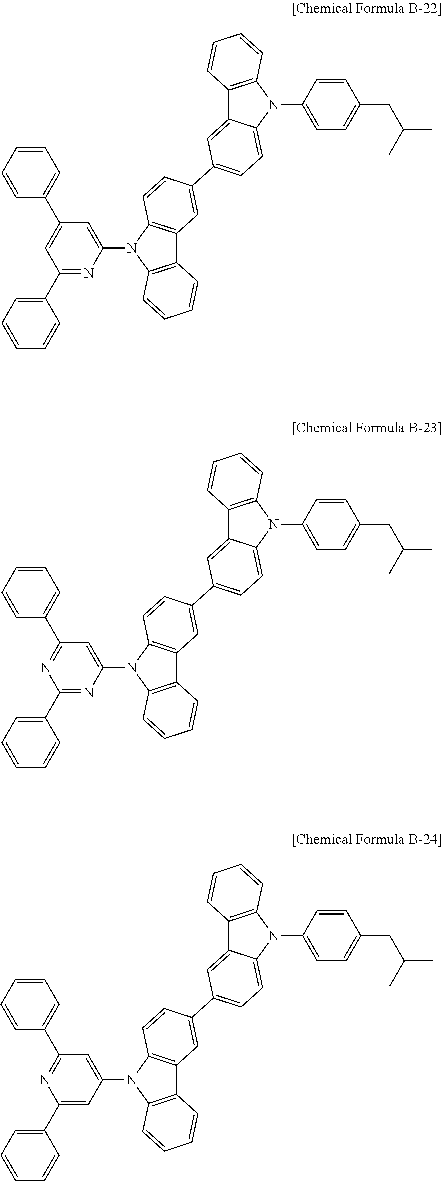 [SMILES: CC(C)Cc1ccc(-n2c3ccccc3c3cc(-c4ccc5c(c4)c4ccccc4n5-c4cc(-c5ccccc5)cc(-c5ccccc5)n4)ccc32)cc1 ; CC(C)Cc1ccc(-n2c3ccccc3c3cc(-c4ccc5c(c4)c4ccccc4n5-c4cc(-c5ccccc5)nc(-c5ccccc5)n4)ccc32)cc1 ; CC(C)Cc1ccc(-n2c3ccccc3c3cc(-c4ccc5c(c4)c4ccccc4n5-c4cc(-c5ccccc5)nc(-c5ccccc5)c4)ccc32)cc1]

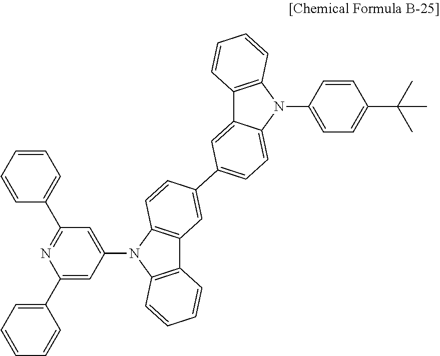 [SMILES: CC(C)(C)c1ccc(-n2c3ccccc3c3cc(-c4ccc5c(c4)c4ccccc4n5-c4cc(-c5ccccc5)nc(-c5ccccc5)c4)ccc32)cc1]

When it is desired that the compound according to an embodiment exhibit both electron properties and hole properties, the functional group with electron properties (e.g., ETU) may be included in the compound to effectively improve life-span of an organic light emitting diode and decrease its driving voltage.

According to the embodiment, the compound for an organic optoelectronic device may have a maximum light emitting wavelength ranging from 320 to 500 nm and triplet excitation energy of 2.0 eV or more (T1), e.g., ranging from 2.0 to 4.0 eV. When the compound exhibits this high excitation energy, the compound may transport a charge to a dopant well and may help improve luminous efficiency of the dopant, and may also decrease the driving voltage by freely regulating HOMO and LUMO energy levels. Accordingly, the compound may be usefully applied as a host material or a charge-transporting material.

The compound for an organic optoelectronic device may be also used as a nonlinear optical material, an electrode material, a chromic material, and as a material applicable to an optical switch, a sensor, a module, a waveguide, an organic transistor, a laser, an optical absorber, a dielectric material, and a membrane due to its optical and electrical properties.

The compound for an organic optoelectronic device according to an embodiment may have a glass transition temperature of 90° C. or higher and a thermal decomposition temperature of 400° C. or higher, so as to help improve thermal stability. Accordingly, it is possible to produce an organic optoelectronic device having a high efficiency.

The compound for an organic optoelectronic device according to an embodiment may play a role in emitting light or injecting and/or transporting electrons, and may act as a light emitting host together with a suitable dopant. For example, the compound for an organic optoelectronic device may be used as a phosphorescent or fluorescent host material, a blue light emitting dopant material, or an electron transporting material.

The compound for an organic optoelectronic device according to an embodiment may be used for an organic thin layer. Thus, the compound may help improve the life span characteristic, efficiency characteristic, electrochemical stability, and thermal stability of an organic optoelectronic device, and may decrease the driving voltage.

According to another embodiment, an organic optoelectronic device is provided. The organic optoelectronic device may include the compound for an organic optoelectronic device according to an embodiment. The organic optoelectronic device may refer to, e.g., an organic photoelectric device, an organic light emitting diode, an organic solar cell, an organic transistor, an organic photo-conductor drum, an organic memory device, or the like. For example, the compound for an organic optoelectronic device according to an embodiment may be included in an electrode or an electrode buffer layer in the organic solar cell to help improve quantum efficiency, and/or may be used as an electrode material for a gate, a source-drain electrode, or the like in the organic transistor.

Hereinafter, a detailed description relating to the organic light emitting diode will be provided.

According to an embodiment, the organic light emitting diode may include an anode, a cathode, and at least one organic thin layer interposed between the anode and the cathode. The at least one organic thin layer may include the compound for an organic optoelectronic device according to an embodiment.

The at least one organic thin layer may include a layer selected from the group of an emission layer, a hole transport layer (HTL), a hole injection layer (HIL), an electron transport layer (ETL), an electron injection layer (EIL), a hole blocking film, and a combination thereof. At least one layer may include the compound for an organic optoelectronic device according to an embodiment. In an implementation, the electron transport layer (ETL) or the electron injection layer (EIL) may include the compound for an organic optoelectronic device according to one embodiment. In an implementation, when the compound for an organic photoelectric device is included in the emission layer, the compound for an organic photoelectric device may be included as a phosphorescent or fluorescent host, e.g., as a fluorescent blue dopant material.

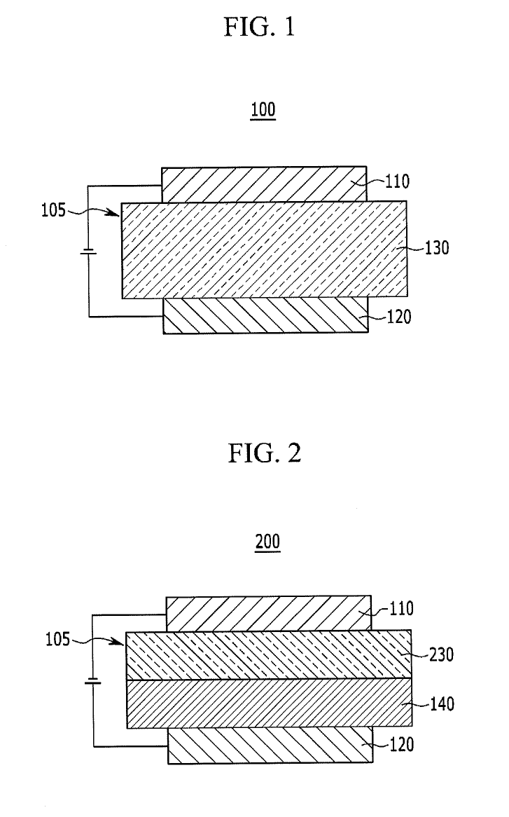

FIGS. 1 to 5 illustrate cross-sectional views showing an organic light emitting diode including the compound for an organic optoelectronic device according to an embodiment.

Referring to FIGS. 1 to 5, organic light emitting diodes 100, 200, 300, 400, and 500 according to an embodiment may include at least one organic thin layer 105 interposed between an anode 120 and a cathode 110.

The anode 120 may include an anode material having a large work function to facilitate hole injection into an organic thin layer. The anode material may include, e.g., a metal such as nickel, platinum, vanadium, chromium, copper, zinc, and gold, or alloys thereof; a metal oxide such as zinc oxide, indium oxide, indium tin oxide (ITO), and indium zinc oxide (IZO); a combined metal and oxide such as ZnO:Al or SnO₂:Sb; or a conductive polymer such as poly(3-methylthiophene), poly[3,4-(ethylene-1,2-dioxy)thiophene] (PEDT), polypyrrole, and polyaniline, but is not limited thereto. In an implementation, a transparent electrode including indium tin oxide (ITO) may be included as an anode.

The cathode 110 may include a cathode material having a small work function to facilitate electron injection into an organic thin layer. The cathode material may include, e.g., a metal such as magnesium, calcium, sodium, potassium, titanium, indium, yttrium, lithium, gadolinium, aluminum, silver, tin, lead, cesium, barium, and the like, or alloys thereof, or a multi-layered material such as LiF/Al, LiO₂/Al, LiF/Ca, LiF/Al, and BaF₂/Ca, but is not limited thereto. In an implementation, a metal electrode including aluminum may be included as a cathode.

Referring to FIG. 1, the organic light emitting diode 100 may include an organic thin layer 105 including only an emission layer 130.

Referring to FIG. 2, a double-layered organic light emitting diode 200 may include an organic thin layer 105 including an emission layer 230 (that includes an electron transport layer (ETL)) and a hole transport layer (HTL) 140. The emission layer 130 may also function as an electron transport layer (ETL), and the hole transport layer (HTL) 140 may have an excellent binding property with a transparent electrode such as ITO or an excellent hole transporting property.

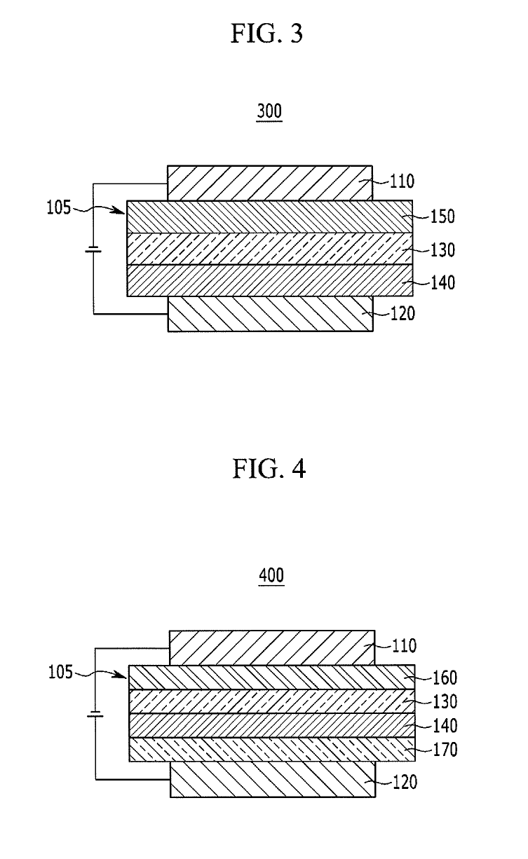

Referring to FIG. 3, a three-layered organic light emitting diode 300 may include an organic thin layer 105 including an electron transport layer (ETL) 150, an emission layer 130, and a hole transport layer (HTL) 140. The emission layer 130 may be independently installed, and layers having an excellent electron transporting property or an excellent hole transporting property may be separately stacked.

As shown in FIG. 4, a four-layered organic light emitting diode 400 may include an organic thin layer 105 including an electron injection layer (EIL) 160, an emission layer 130, a hole transport layer (HTL) 140, and a hole injection layer (HIL) 170 for binding with the anode 120 of ITO.

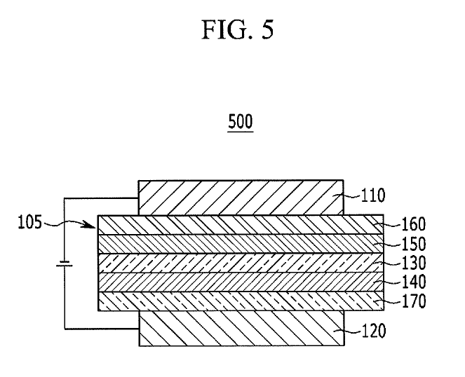

As shown in FIG. 5, a five layered organic light emitting diode 500 may include an organic thin layer 105 including an electron transport layer (ETL) 150, an emission layer 130, a hole transport layer (HTL) 140, and a hole injection layer (HIL) 170, and may further include an electron injection layer (EIL) 160 to achieve a low voltage.

In FIG. 1 to FIG. 5, the organic thin layer 105 including at least one selected from the group of an electron transport layer (ETL) 150, an electron injection layer (EIL) 160, an emission layer 130 and 230, a hole transport layer (HTL) 140, a hole injection layer (HIL) 170, and combinations thereof may include the compound for an organic optoelectronic device. The compound for an organic optoelectronic device may be used for an electron transport layer (ETL) 150 or electron injection layer (EIL) 160. When it is used for the electron transport layer (ETL), it is possible to provide an organic light emitting diode having a simpler structure because it may not require an additional hole blocking layer (not shown).

Furthermore, when the compound for an organic optoelectronic device is included in the emission layers 130 and 230, the material for an organic optoelectronic device may be included as a phosphorescent or fluorescent host or a fluorescent blue dopant.

The organic light emitting diode may be fabricated by: forming an anode on a substrate; forming an organic thin layer in accordance with a dry coating method such as evaporation, sputtering, plasma plating, and ion plating, or a wet coating method such as spin coating, dipping, and flow coating; and providing a cathode thereon.

Another embodiment provides a display device including the organic light emitting diode according to the above embodiment.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### Preparation of Compound for an Organic Optoelectronic Device

### Example 1

### Compound A-1

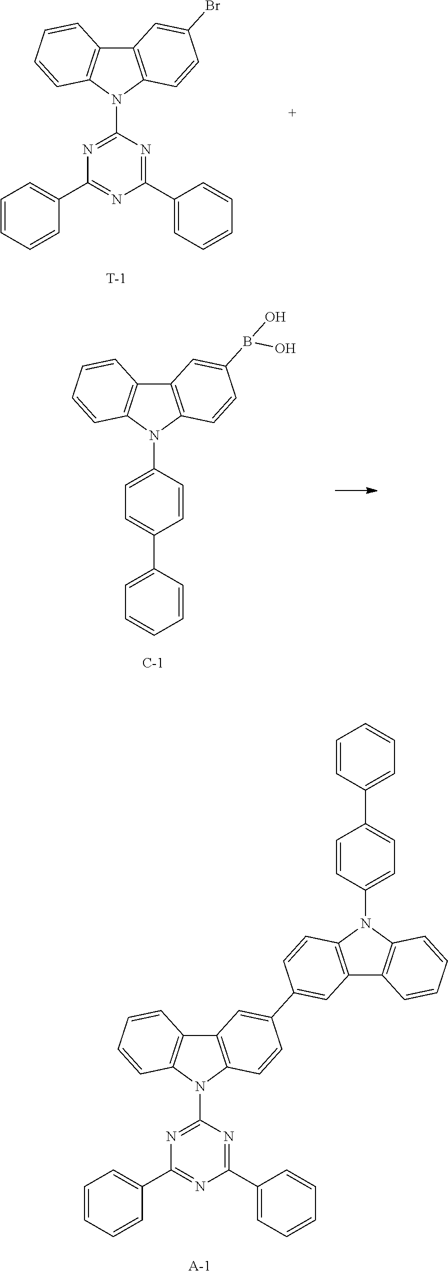

9.6 g of an intermediate compound T-1 and 8.7 g of an intermediate compound C-1 were dissolved in 100 ml of tetrahydrofuran in a 250 ml round-bottomed flask with a thermometer, a reflux-condenser, and an agitator under a nitrogen atmosphere. 80 ml of a 2M-potassium carbonate aqueous solution was added thereto.

Next, 1.2 g of tetrakis(triphenylphosphine) palladium was added to the mixture, and the resulting mixture was refluxed for 12 hours. When the reaction was complete, the reactant was extracted several times with methylene chloride. The extract was treated with anhydrous sulfuric acid magnesium to remove moisture. Then, the resulting product was filtered, and a solvent therein was removed.

Then, the reactant was recrystallized for purification, obtaining 10.0 g of a compound A-1. The synthesized compound A-1 was evaluated with LC-Mass Spec and was identified to have 717.42 of a [M+H]⁺ molecular weight.

### Example 2

### Compound A-3

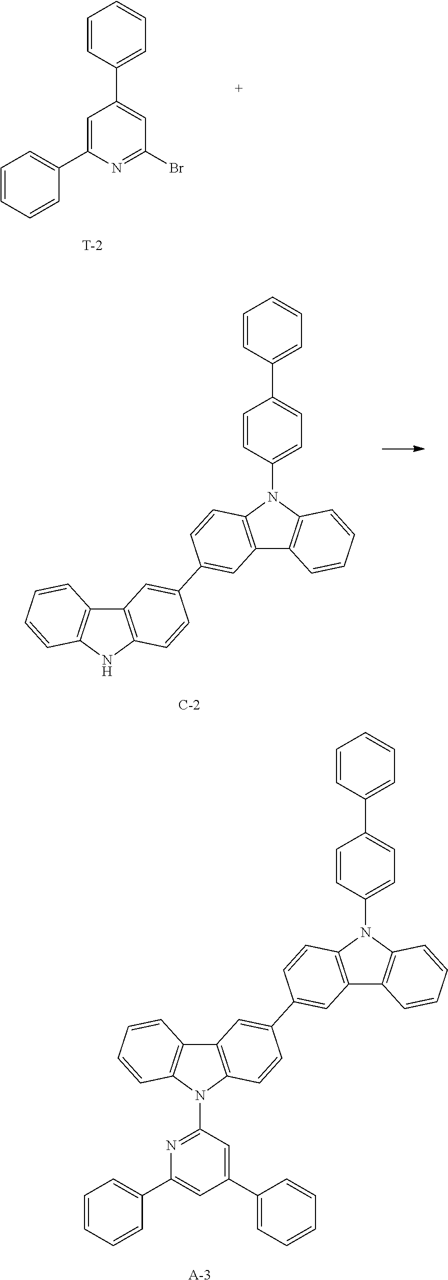

7.4 g of an intermediate compound T-2 and 9.7 g of an intermediate compound C-2 were dissolved in 100 ml of tetrahydrofuran in a 25 0 ml round-bottomed flask with a thermometer, a reflux-condenser, and an agitator under a nitrogen atmosphere. 0.3 g of sodium tert-butoxide, 0.9 g of palladium dibenzylideneamine, and 0.4 g of phosphorus tertiarybutyl were added thereto. The mixture was refluxed for 12 hours. When the reaction was complete, the reactant was extracted several time with methylene chloride and then, treated with anhydrous sulfuric acid magnesium to remove moisture. The resulting reactant was filtered, and a solvent therein was removed.

Next, the reactant was recrystallized for purification, obtaining 10.7 g of a compound A-3. The synthesized compound A-3 was evaluated with LC-Mass Spec and was identified to have 715.31 of a [M+H]⁺ molecular weight.

### Example 3

### Compound A-7

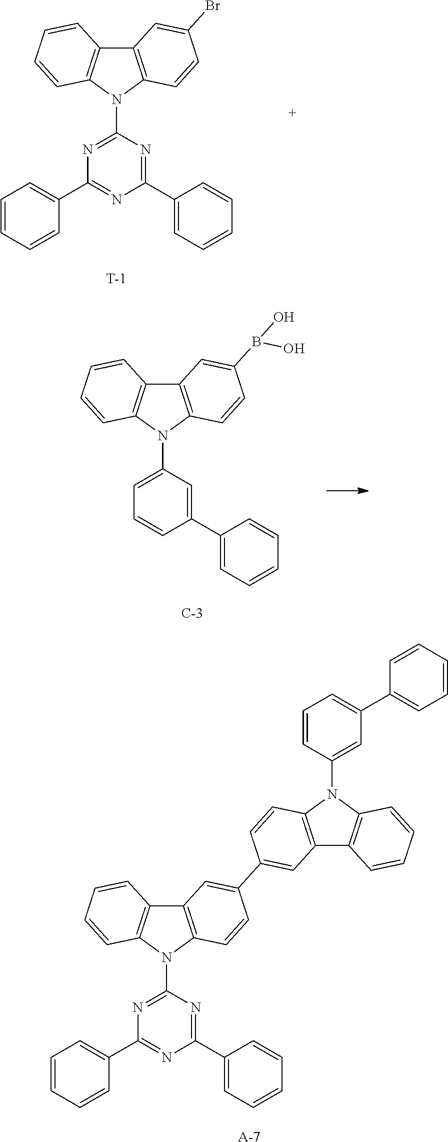

9.6 g of an intermediate compound T-1 and 8.7 g of an intermediate compound C-3 were dissolved in 100 ml of tetrahydrofuran in a 250 ml round-bottomed flask with a thermometer, a reflux-condenser, an agitator under a nitrogen atmosphere. 80 ml of a 2M-potassiumcarbonate aqueous solution was added thereto. Next, 1.2 g of tetrakis(triphenylphosphine)palladium was added to the mixture. The resulting mixture was refluxed for 12 hours. When the reaction was complete, the reactant was extracted several times with methylene chloride and treated with anhydrous sulfuric acid magnesium to remove moisture and then, filtered. Then, a solvent therein was removed from the filtrated product.

Then, the reactant was recrystallized for purification, obtaining 10.7 g of a compound A-7. The synthesized compound A-7 was evaluated with LC-Mass Spec and was identified to have 717.42 of a [M+H]⁺ molecular weight.

### Example 4

### Compound A-8

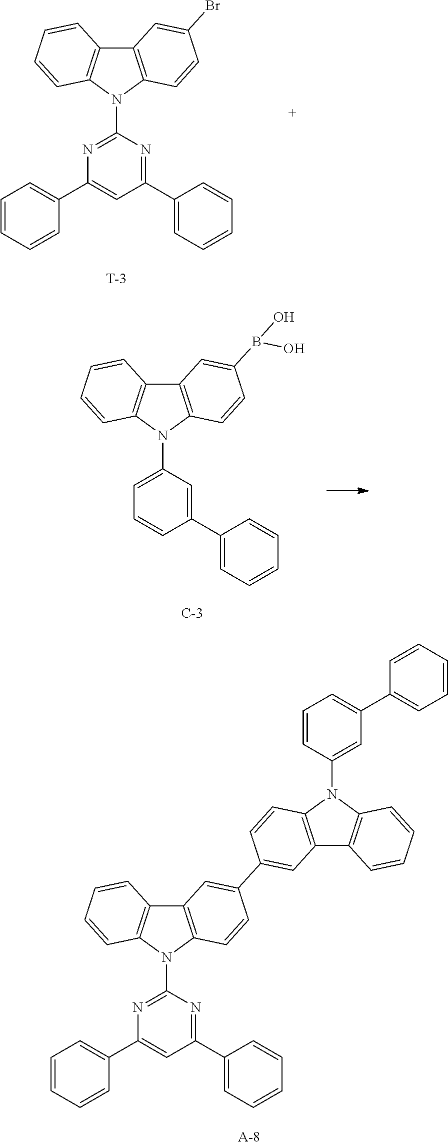

9.5 g of an intermediate compound T-3 and 8.7 g of an intermediate compound C-3 were dissolved in 100 ml of tetrahydrofuran in a 250 ml round-bottomed flask with a thermometer, a reflux-condenser, and an agitator under a nitrogen atmosphere. 80 ml of a 2M-potassiumcarbonate aqueous solution was added thereto. Next, 1.2 g of tetrakis(triphenylphosphine)palladium was added to the mixture. The resulting mixture was refluxed for 12 hours. When the reaction was complete, the reactant was extracted several times with methylene chloride, treated with anhydrous sulfuric acid magnesium to remove moisture, and then, filtered. Then, a solvent therein was removed.

The resulting reactant was recrystallized for purification, obtaining 12.1 g of a compound A-8. The synthesized compound A-8 was evaluated with LC-Mass Spec and was identified to have 715.86 of a [M+H]⁺ molecular weight.

### Example 5

### Compound B-1

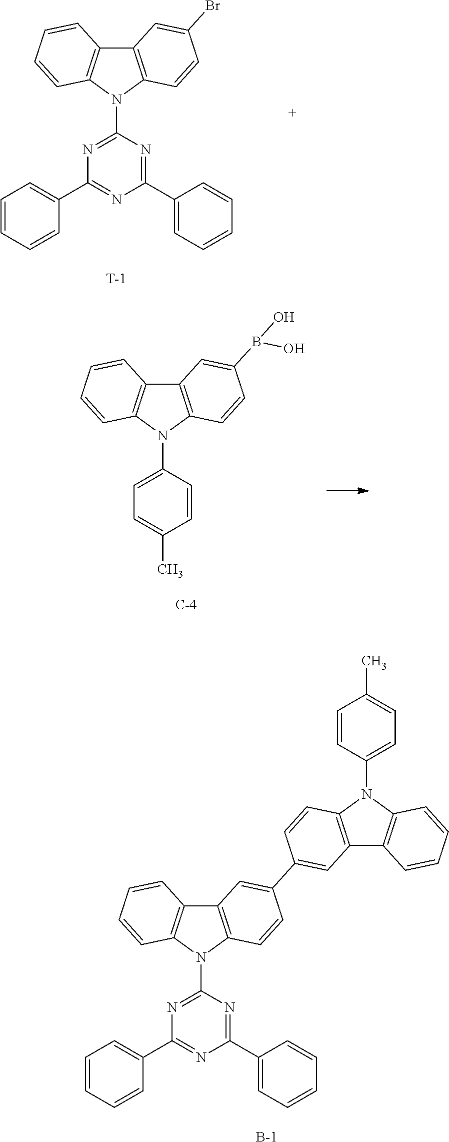

9.5 g of an intermediate compound T-3 and 7.2 g of an intermediate compound C-4 were dissolved in 100 ml of tetrahydrofuran in a 250 ml round-bottomed flask with a thermometer, a reflux-condenser, and an agitator under a nitrogen atmosphere. 80 ml of a 2M-potassiumcarbonate aqueous solution was added thereto. Next, 1.2 g of tetrakis(triphenylphosphine)palladium was added to the mixture. The resulting mixture was refluxed for 12 hours. When the reaction was complete, the reactant was extracted several times with methylene chloride and treated with anhydrous sulfuric acid magnesium to remove moisture, filtered, and then, a solvent therein was removed.

The resulting reactant was purified by performing column chromatography and recrystallization, obtaining 8.5 g of a compound B-1. The synthesized compound B-1 was evaluated with LC-Mass Spec and was identified to have 654.74 of a [M+H]⁺ molecular weight.

### Example 6

### Compound B-2

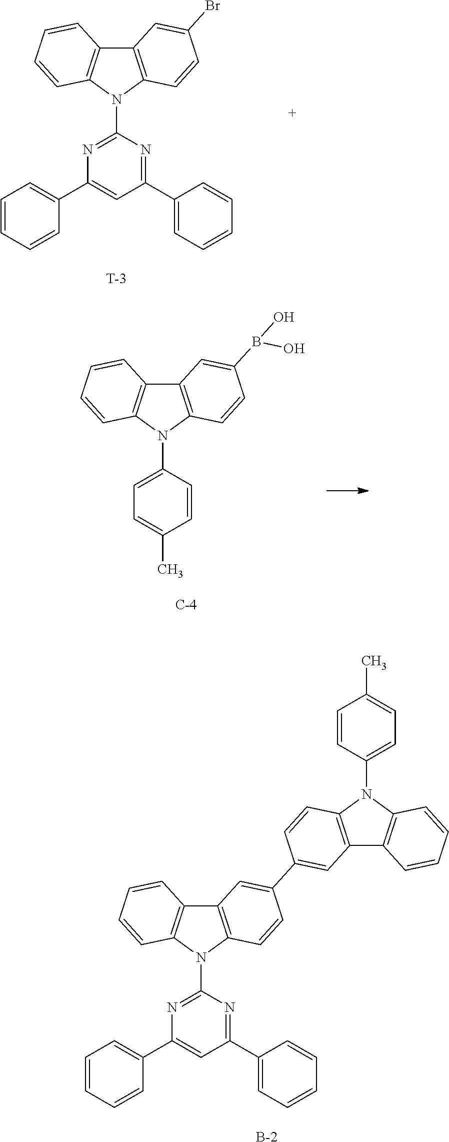

9.0 g of an intermediate compound T-3 and 7.2 g of an intermediate compound C-4 were dissolved in 100 ml of tetrahydrofuran in a 250 ml round-bottomed flask with a thermometer, a reflux-condenser, and an agitator under a nitrogen atmosphere. 80 ml of a 2M-potassiumcarbonate aqueous solution was added thereto. Next, 1.2 g of tetrakis(triphenylphosphine)palladium was added to the mixture. The resulting mixture was refluxed for 12 hours. When the reaction was complete, the reactant was extracted several times with methylene chloride, treated with anhydrous sulfuric acid magnesium to remove moisture, and filtered, and then, a solvent therein was removed.

The resulting reactant was purified by performing column chromatography and recrystallization, obtaining 9.2 g of a compound B-2. The synthesized compound B-2 was evaluated with LC-Mass Spec and was identified to have 654.74 of a [M+H]⁺ molecular weight.

### Comparative Example 1

### Compound R-1

A compound represented by the following Chemical Formula R-1 was prepared.

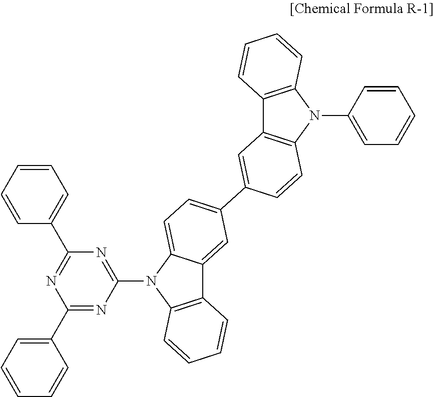 [SMILES: c1ccc(-c2nc(-c3ccccc3)nc(-n3c4ccccc4c4cc(-c5ccc6c(c5)c5ccccc5n6-c5ccccc5)ccc43)n2)cc1]

### Comparative Example 2

### Compound R-2

A compound represented by the following Chemical Formula R-2 was prepared.

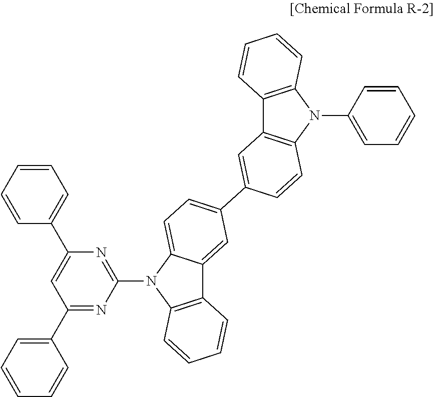 [SMILES: c1ccc(-c2cc(-c3ccccc3)nc(-n3c4ccccc4c4cc(-c5ccc6c(c5)c5ccccc5n6-c5ccccc5)ccc43)n2)cc1]

### Comparative Example 3

### Compound R-3

A compound represented by the following Chemical Formula R-3 was prepared.

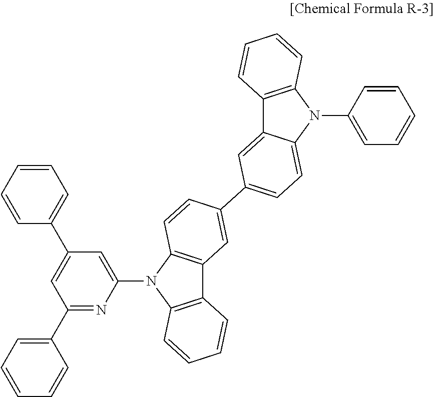 [SMILES: c1ccc(-c2cc(-c3ccccc3)nc(-n3c4ccccc4c4cc(-c5ccc6c(c5)c5ccccc5n6-c5ccccc5)ccc43)c2)cc1]

(Result of Energy Level Calculation)

The compounds synthesized according to Examples 1 to 6 and Comparative Examples 1 to 3 were calculated regarding energy level with Gaussian 03 (b3lyp/6-31 g). The results are provided in Table 1.

TABLE 1

| | | | HOMO | LUMO | ΔE | ΔE |
|---|---|---|---|---|---|---|
| Material | ETU | R | (eV) | (eV) | (T1) | (S1) |
| Comparative | triazinyl | hydrogen | −5.16 | −1.89 | 2.83 | 2.90 |
| Example 1 | group | | | | | |
| (R-1) | | | | | | |
| Example 1 | triazinyl | p-phenyl | −5.14 | −1.89 | 2.83 | 2.89 |
| (A-1) | group | | | | | |
| Example 3 | triazinyl | m-phenyl | −5.15 | −1.89 | 2.82 | 2.89 |
| (A-7) | group | | | | | |
| Example 5 | triazinyl | p-methyl | −5.12 | −1.88 | 2.82 | 2.88 |
| (B-1) | group | | | | | |
| Comparative | pyrimidinyl | hydrogen | −5.05 | −1.72 | 2.78 | 2.93 |
| Example 2 | group | | | | | |
| (R-2) | | | | | | |
| Example 4 | pyrimidinyl | m-phenyl | −5.03 | −1.77 | 2.71 | 2.84 |
| (A-8) | group | | | | | |
| Example 6 | pyrimidinyl | p-methyl | −5.00 | −1.76 | 2.71 | 2.84 |
| (B-2) | group | | | | | |
| Comparative | pyridinyl | hydrogen | −4.97 | −1.45 | 2.91 | 3.09 |
| Example 3 | group | | | | | |
| (R-3) | | | | | | |
| Example 2 | pyridinyl | p-phenyl | −4.97 | −1.46 | 2.91 | 3.08 |
| (A-3) | group | | | | | |

Referring to Table 1, ETU indicates the ETU substituent of Chemical Formula 1, and the R represents at least substituent among R¹ to R⁵ in the above Chemical Formula 1.

As may be seen in Table 1, the compounds according to the Examples exhibited different energy level characteristic depending on ETU of the materials but not on kinds of R and location of a substituent.

### Preparation of Organic Light Emitting Diode

### Example 7

### Preparation of Organic Light Emitting Diode

A glass substrate having a 1,500 Å-thick ITO (Indium tin oxide) layer was cleaned with ultrasonic waves using distilled water. Next, the resulting substrate was cleaned with ultrasonic waves using a solvent such as isopropyl alcohol, acetone, methanol, or the like, and dried. The dried substrate was moved to a plasma cleaner and cleaned by using an oxygen plasma for 5 minutes there and then, moved to a vacuum depositor. The ITO transparent electrode was used as an anode, and the following HTM compound was vacuum-deposited to form a 1,200 Å-thick hole injection layer (HIL).

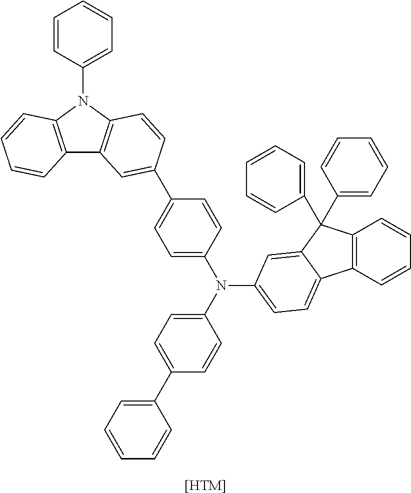

On the hole transport layer (HTL), a 300 Å-thick emission layer was formed by doping the material synthesized in Example 1 as a host with 7 wt % of the following PhGD compound as a green phosphorescent dopant and vacuum-depositing the doped material.

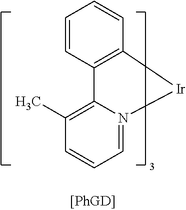

Next, electron transport layer (ETL) was formed on the emission layer by laminating the following BAlq [bis(2-methyl-8-quinolinolato-N1,O8)-(1,1′-biphenyl-4-olato)aluminum] compound to be 50 Åthick and sequentially, the following Alq₃ [tris(8-hydroxyquinolinato)aluminium] compound to be 250 Åthick thereon. Then, 5 Å-thick LiF and 1,000 Å-thick Al were sequentially vacuum-deposited on the electron transport layer (ETL), fabricating a cathode and thus, an organic light emitting diode.

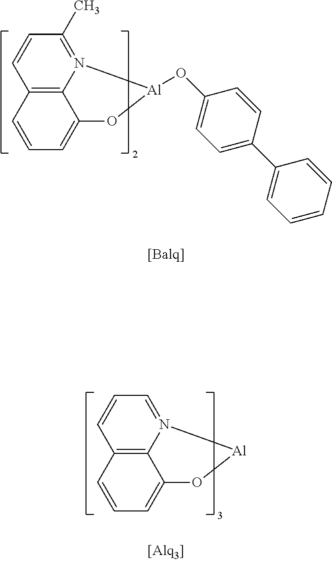

### Example 8

An organic light emitting diode was fabricated according to the same method as Example 7 except for using the compound of Example 2 instead of the compound of Example 1.

### Example 9

An organic light emitting diode was fabricated according to the same method as Example 7 except for using the compound of Example 3 instead of the compound of Example 1.

### Example 10

An organic light emitting diode was fabricated according to the same method as Example 7 except for using the compound of Example 4 instead of the compound of Example 1.

### Example 11

An organic light emitting diode was fabricated according to the same method as Example 7 except for using the compound of Example 5 instead of the compound of Example 1.

### Example 12

An organic light emitting diode was fabricated according to the same method as Example 7 except for using the compound of Example 6 instead of the compound of Example 1.

### Comparative Example 4

An organic light emitting diode was fabricated according to the same method as Example 7 except for using the compound of Comparative Example 1 instead of the compound of Example 1 as a host.

### Comparative Example 5

An organic light emitting diode was fabricated according to the same method as Example 7 except for using the compound of Comparative Example 2 instead of the compound of Example 1 as a host.

### Comparative Example 6

An organic light emitting diode was fabricated according to the same method as Example 7 except for using the compound of Comparative Example 3 instead of the compound of Example 1 as a host.

(Performance Evaluation of Organic Light Emitting Diode)

Each organic light emitting diode according to Examples 7 to 12 and Comparative Examples 4 to 6 was measured regarding current density change, luminance change, and luminous efficiency depending on a voltage.

(1) Current Density Change Depending on Voltage Change

The organic light emitting diodes were measured regarding current by increasing a voltage from 0 V to 10 V with a current-voltage meter (Keithley 2400). The current was divided with an area.

(2) Luminance Change Depending on Voltage Change

The organic light emitting diodes were measured regarding luminance by increasing a voltage from 0 V to 10 V with a luminance meter (Minolta Cs-1000A).

(3) Luminous Efficiency Measurement

The luminance and current density obtained in the above (1) and (2) and a voltage were used to calculate current efficiency (cd/A) at the same current density (10 mA/cm²).

(4) Device Life-Span Measurement

The time it took for luminance to drop from 3,000 cd/m² to 2,910 cd/m² by 3% was measured.

The following Table 2 shows the device evaluation result of compounds that included a triazinyl group as ETU in the above Chemical Formula 1.

TABLE 2

| | | | | | | | | Life- |
|---|---|---|---|---|---|---|---|---|
| | | | | | | | | span |
| | Host | Vd | Cd/A | lm/W | cd/m² | CIEx | CIEy | (h) |
| Comparative | R-1 | 5.21 | 53.5 | 32.3 | 3,000 | 0.338 | 0.623 | 10 |
| Example 4 | | | | | | | | |
| Example 7 | A-1 | 5.13 | 60.7 | 37.2 | 3,000 | 0.340 | 0.622 | 27 |
| Example 11 | B-1 | 5.11 | 62.4 | 38.4 | 3,000 | 0.337 | 0.624 | 130 |
| Example 9 | A-7 | 5.32 | 64.9 | 38.4 | 3,000 | 0.340 | 0.621 | 100 |

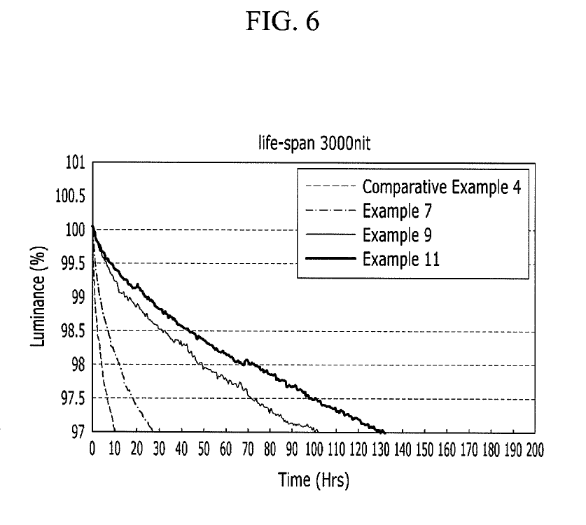

FIG. 6 illustrates a graph showing life-span data of the light emitting diodes according to Examples 7, 9, and 11 and Comparative Example 4.

The light emitting diodes including an emission layer prepared by applying compounds A-1, B-1, and A-7 according to the Examples as a host exhibited improved efficiency and life-span, compared with the light emitting diodes an emission layer prepared by applying compound R-1 according to Comparative Example 1 as a host.

The following Table 3 shows evaluation results of the devices that included a compound having a pyrimidinyl group as ETU in the above Chemical Formula 1.

TABLE 3

| | | | | | | | | Life- |
|---|---|---|---|---|---|---|---|---|
| | | | | | | | | span |
| | Host | Vd | Cd/A | lm/W | cd/m² | CIEx | CIEy | (h) |
| Comparative | R-2 | 5.04 | 58.4 | 36.4 | 3,000 | 0.341 | 0.621 | 4 |
| Example 5 | | | | | | | | |
| Example 12 | B-2 | 4.91 | 66.3 | 42.5 | 3,000 | 0.339 | 0.623 | 37 |
| Example 10 | A-8 | 4.84 | 66.6 | 43.3 | 3,000 | 0.340 | 0.621 | 78 |

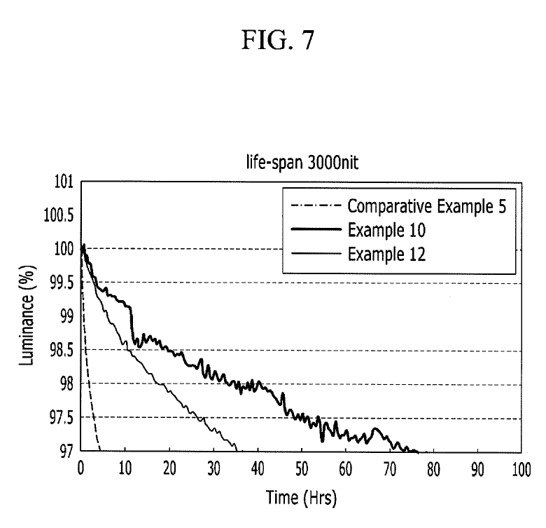

FIG. 7 illustrates a graph showing life-span data of the organic light emitting diodes according to Examples 10 and 12 and Comparative Example 5.

The organic light emitting diodes including an emission layer using compounds B-2 and A-8 as a host according to the Examples exhibited improved efficiency and life-span, compared with the organic light emitting diode including an emission layer using compound R-2 as a host according to Comparative Example 2.

The following Table 4 shows the device evaluation results of the devices that included a compound having a pyridinyl group as ETU in the above Chemical Formula 1.

TABLE 4

| | | | | | | | | Life- |
|---|---|---|---|---|---|---|---|---|
| | | | | | | | | span |
| | Host | Vd | Cd/A | lm/W | cd/m² | CIEx | CIEy | (h) |
| Comparative | R-3 | 5.14 | 58.4 | 35.7 | 3,000 | 0.335 | 0.624 | 20 |
| Example 6 | | | | | | | | |
| Example 8 | A-3 | 5.02 | 59.9 | 37.5 | 3,000 | 0.339 | 0.622 | 35 |

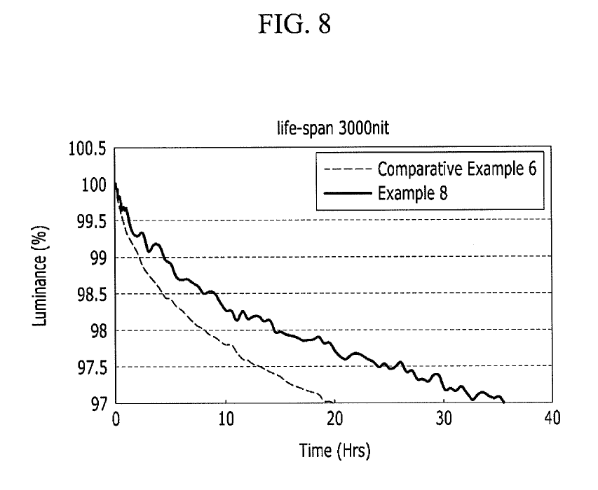

FIG. 8 illustrates a graph showing life-span data of the organic light emitting diodes according to Example 8 and Comparative Example 6.

The organic light emitting diode including an emission layer formed by applying compound A-3 as a host according to an Example exhibited improved efficiency and life-span, compared with the organic light emitting diode including an emission layer formed by applying compound R-3 as a host according to Comparative Example 3.

Based on the energy level characteristic evaluation in Table 1 and the device evaluations in Tables 2 to 4, when an alkyl group or an aryl group other than hydrogen was added to a phenyl group substituted for bicarbazole in a derivative with similar energy level characteristic, a device exhibited improved performance and particularly, increased life-span.

By way of summation and review, a phosphorescent light emitting material may be used for a light emitting material of an organic light emitting diode, in addition to the fluorescent light emitting material. Such a phosphorescent material emits lights by transiting the electrons from a ground state to an exited state, non-radiance transiting of a singlet exciton to a triplet exciton through intersystem crossing, and transiting a triplet exciton to a ground state to emit light.

As described above, in an organic light emitting diode, an organic material layer may include a light emitting material and a charge transport material, e.g., a hole injection material, a hole transport material, an electron transport material, an electron injection material, or the like.

The light emitting material may be classified as blue, green, and/or red light emitting materials according to emitted colors, and yellow and orange light emitting materials to emit colors approaching natural colors.

When one material is used as a light emitting material, a maximum light emitting wavelength may be shifted to a long wavelength or color purity may decrease because of interactions between molecules, or device efficiency may decrease because of a light emitting quenching effect. Thus, a host/dopant system may be included as a light emitting material in order to help improve color purity and help increase luminous efficiency and stability through energy transfer.

In order to implement excellent performance of an organic light emitting diode, a material constituting an organic material layer, e.g., a hole injection material, a hole transport material, a light emitting material, an electron transport material, an electron injection material, and a light emitting material such as a host and/or a dopant, should be stable and have good efficiency. Thus, development of an organic material layer forming material for an organic light emitting diode may be desirable. This material development may also be suitable for other organic optoelectric devices.

A low molecular organic light emitting diode may be manufactured as a thin film in a vacuum deposition method and may have good efficiency and life-span performance. A polymer organic light emitting diode may be manufactured in an Inkjet or spin coating method and may have an advantage of low initial cost and being large-sized. A low molecular material using a solution process may exhibit better performance than a polymer material, and thus development for a low molecular material have been considered.

Both low molecular organic light emitting and polymer organic light emitting diodes may have an advantage of self-light emitting, high speed response, wide viewing angle, ultrathin, high image quality, durability, large driving temperature range, or the like. For example, they may have good visibility due to self-light emitting characteristic, compared with an LCD (liquid crystal display), and may have an advantage of decreasing thickness and weight (compared to LCDs) up to a third, because a backlight is not required.

In addition, they may have a response speed 1,000 times faster than LCDs. Thus, they may realize a perfect motion picture without after-image. Based on these advantages, they have been remarkably developed to have 80 times efficiency and more than 100 times life-span since they come out for the first time in the late 1980s. Recently, they have rapidly been becoming larger, such as a 40-inch organic light emitting diode panel.

Organic light emitting diodes should simultaneously have improved luminous efficiency and life-span in order to be larger. Herein, their luminous efficiency may need smooth combination between holes and electrons in an emission layer. However, an organic material in general may have slower electron mobility than hole mobility. Thus, inefficient combination between holes and electrons may occur. Accordingly, increasing electron injection and mobility from a cathode and simultaneously preventing movement of holes may be desirable.

In order to improve life-span, a material crystallization caused by Joule heat (generated during device operation) should be prevented. Accordingly, an organic compound having excellent electron injection and mobility, and high electrochemical stability may be desirable.

The organic optoelectronic device according to an embodiment may have excellent life-span, efficiency, electrochemical stability, and thermal stability.

The embodiments provide a compound for an organic optoelectronic device that may act as a hole injection and hole transport, or an electron injection and transport, and also act as a light emitting host along with an appropriate dopant.

The embodiments provide a light emitting diode which may have excellent life span, efficiency, a driving voltage, electrochemical stability, and thermal stability.

The compound for an organic optoelectronic device may have an excellent hole or electron transporting property, high film stability, thermal stability, and triplet excitation energy.

The compound may be used as a hole injection/transport material of an emission layer, a host material, or an electron injection/transport material. The organic optoelectronic device may have an excellent electrochemical and thermal stability, and therefore, may provide an organic light emitting diode having an excellent life-span characteristic, and high luminous efficiency at a low driving voltage.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device, the compound being represented by the following Chemical Formula 1: 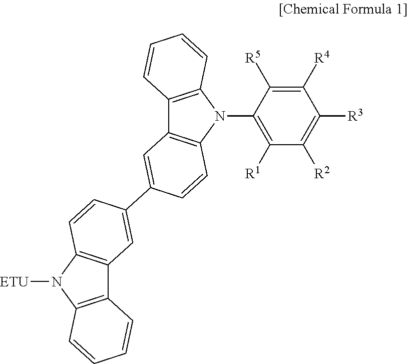
wherein, in Chemical Formula 1,
ETU is a substituent having an electron property and is a substituted or unsubstituted C2 to C30 heteroaryl group, and
R¹ to R⁵ are each independently hydrogen, deuterium, a substituted or unsubstituted C1 to C30 alkyl group, a substituted or unsubstituted C6 to C36 aryl group, or a combination thereof, provided that at least one of R¹ to R⁵ is a substituted or unsubstituted C1 to C30 alkyl group or a substituted or unsubstituted C6 to C36 aryl group.

2. The compound for an organic optoelectronic device as claimed in claim 1, wherein at least one of R² or R⁴ is a substituted or unsubstituted C1 to C30 alkyl group or a substituted or unsubstituted C6 to C36 aryl group.

3. The compound for an organic optoelectronic device as claimed in claim 2, wherein at least one of R² or R⁴ is a substituted or unsubstituted phenyl group.

4. The compound for an organic optoelectronic device as claimed in claim 1, wherein at least one of R² to R⁴ is a substituted or unsubstituted methyl group.

5. The compound for an organic optoelectronic device as claimed in claim 1, wherein the ETU is a substituted or unsubstituted pyridinyl group, a substituted or unsubstituted pyrimidinyl group, a substituted or unsubstituted triazinyl group, or a combination thereof.

6. The compound for an organic optoelectronic device as claimed in claim 5, wherein the ETU is a substituent represented by one of the following Chemical Formulae 2 to 6, in which * represents a bonding location with a nitrogen atom of Chemical Formula 1:

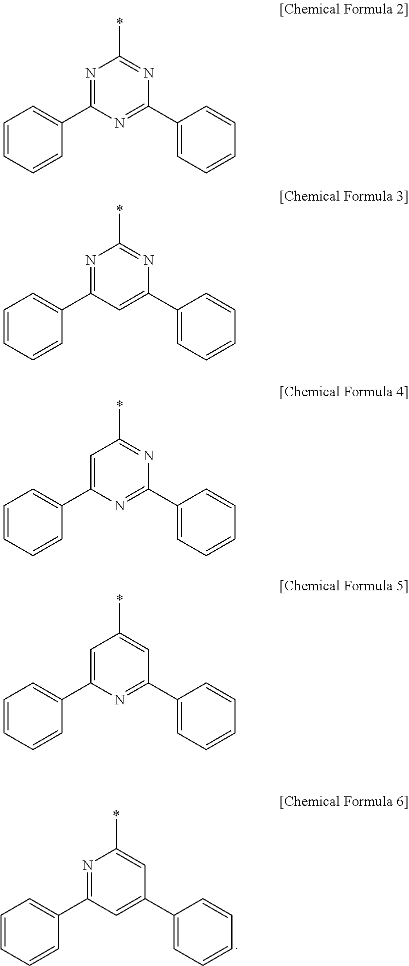 [SMILES: *c1nc(-c2ccccc2)nc(-c2ccccc2)n1 ; *c1nc(-c2ccccc2)cc(-c2ccccc2)n1 ; *c1cc(-c2ccccc2)nc(-c2ccccc2)n1 ; *c1cc(-c2ccccc2)nc(-c2ccccc2)c1 ; *c1cc(-c2ccccc2)cc(-c2ccccc2)n1]

7. The compound for an organic optoelectronic device as claimed in claim 1, wherein the compound for an organic optoelectronic device is represented by one of the following Chemical Formulae A-1 to A-39:

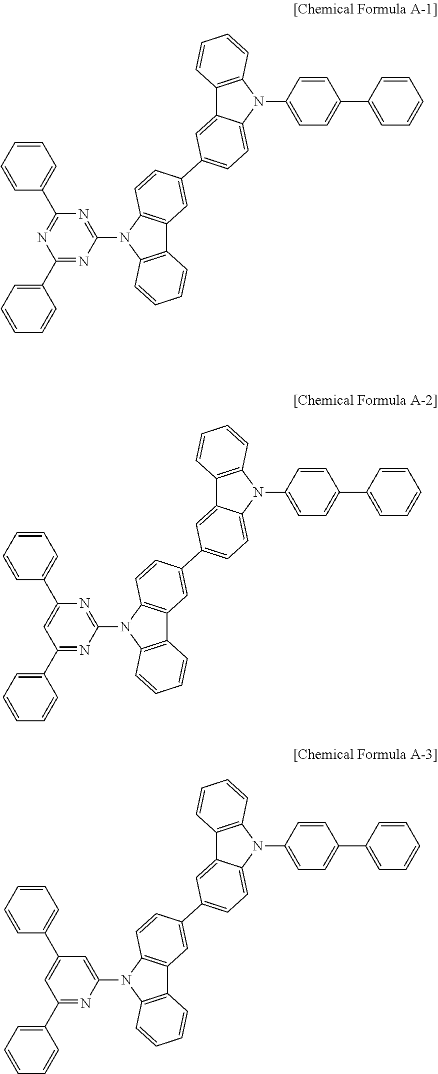 [SMILES: c1ccc(-c2ccc(-n3c4ccccc4c4cc(-c5ccc6c(c5)c5ccccc5n6-c5nc(-c6ccccc6)nc(-c6ccccc6)n5)ccc43)cc2)cc1 ; c1ccc(-c2ccc(-n3c4ccccc4c4cc(-c5ccc6c(c5)c5ccccc5n6-c5nc(-c6ccccc6)cc(-c6ccccc6)n5)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)n5)ccc43)cc2)cc1]

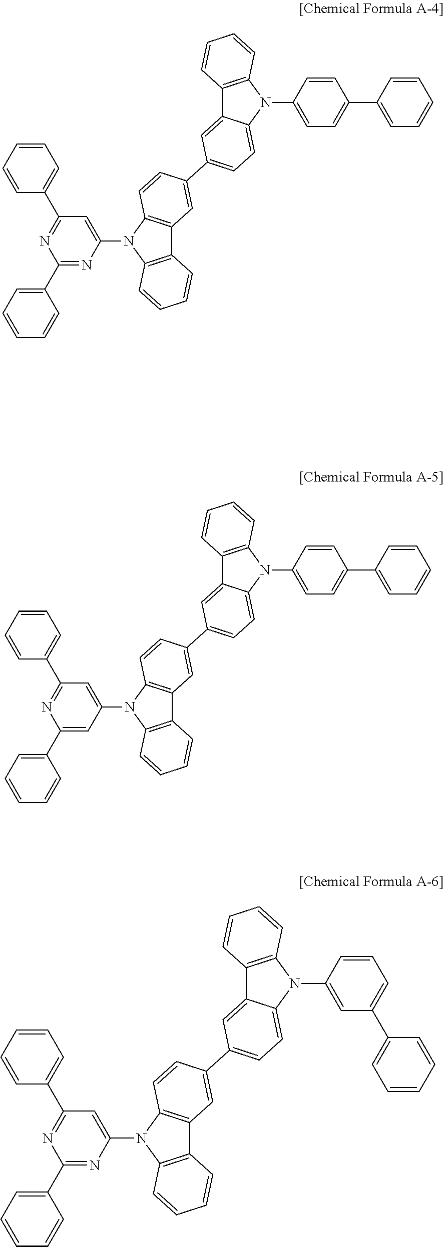 [SMILES: c1ccc(-c2ccc(-n3c4ccccc4c4cc(-c5ccc6c(c5)c5ccccc5n6-c5cc(-c6ccccc6)nc(-c6ccccc6)n5)ccc43)cc2)cc1 ; c1ccc(-c2ccc(-n3c4ccccc4c4cc(-c5ccc6c(c5)c5ccccc5n6-c5cc(-c6ccccc6)nc(-c6ccccc6)c5)ccc43)cc2)cc1 ; c1ccc(-c2cccc(-n3c4ccccc4c4cc(-c5ccc6c(c5)c5ccccc5n6-c5cc(-c6ccccc6)nc(-c6ccccc6)n5)ccc43)c2)cc1]

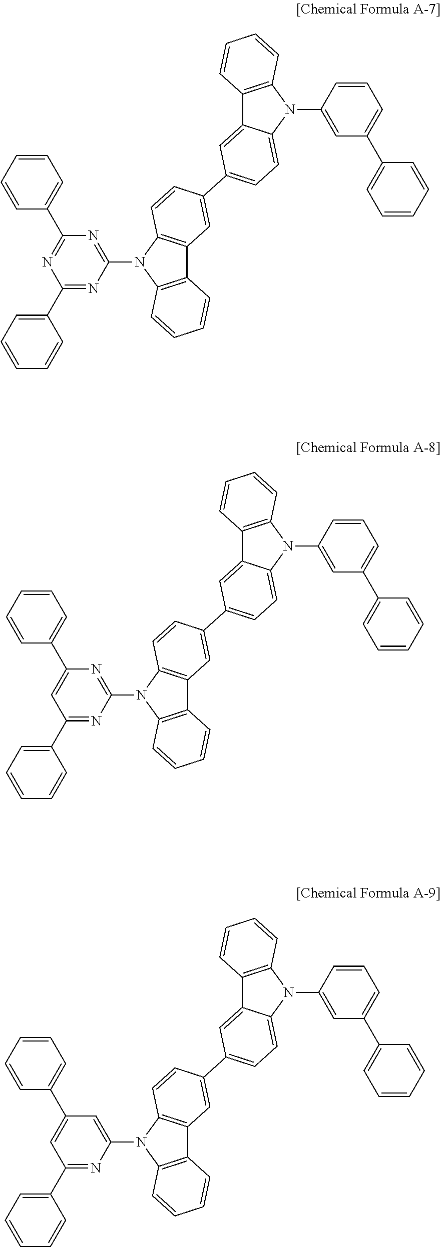 [SMILES: c1ccc(-c2cccc(-n3c4ccccc4c4cc(-c5ccc6c(c5)c5ccccc5n6-c5nc(-c6ccccc6)nc(-c6ccccc6)n5)ccc43)c2)cc1 ; c1ccc(-c2cccc(-n3c4ccccc4c4cc(-c5ccc6c(c5)c5ccccc5n6-c5nc(-c6ccccc6)cc(-c6ccccc6)n5)ccc43)c2)cc1 ; c1ccc(-c2cccc(-n3c4ccccc4c4cc(-c5ccc6c(c5)c5ccccc5n6-c5cc(-c6ccccc6)cc(-c6ccccc6)n5)ccc43)c2)cc1]

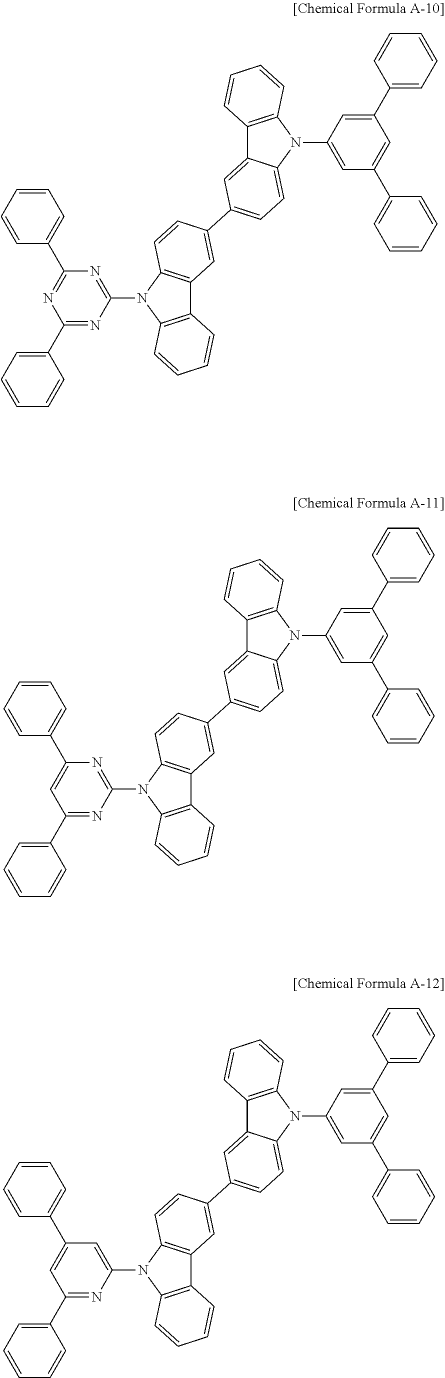 [SMILES: c1ccc(-c2cc(-c3ccccc3)cc(-n3c4ccccc4c4cc(-c5ccc6c(c5)c5ccccc5n6-c5nc(-c6ccccc6)nc(-c6ccccc6)n5)ccc43)c2)cc1 ; c1ccc(-c2cc(-c3ccccc3)cc(-n3c4ccccc4c4cc(-c5ccc6c(c5)c5ccccc5n6-c5nc(-c6ccccc6)cc(-c6ccccc6)n5)ccc43)c2)cc1 ; c1ccc(-c2cc(-c3ccccc3)cc(-n3c4ccccc4c4cc(-c5ccc6c(c5)c5ccccc5n6-c5cc(-c6ccccc6)cc(-c6ccccc6)n5)ccc43)c2)cc1]

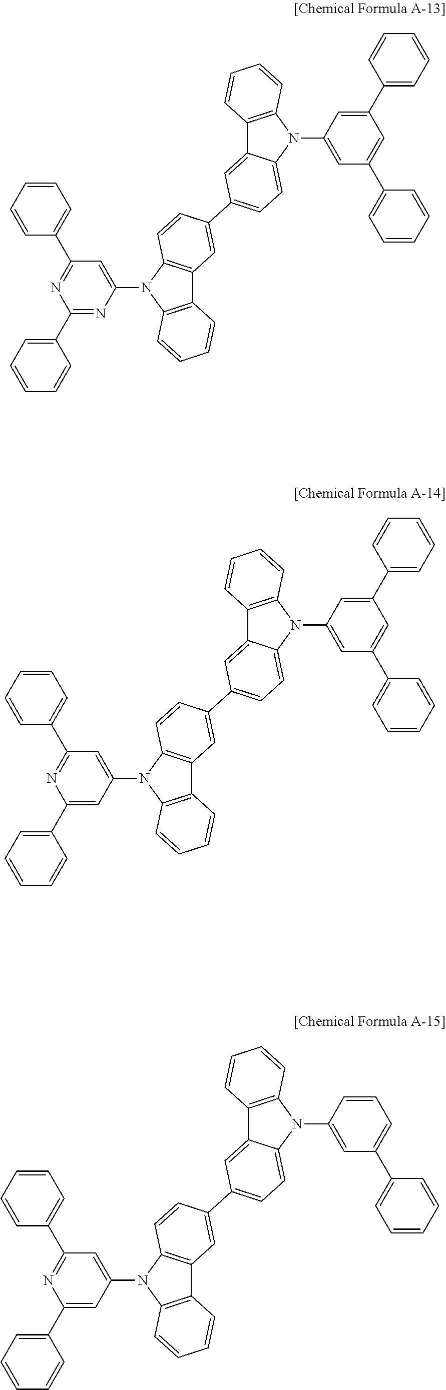 [SMILES: c1ccc(-c2cc(-c3ccccc3)cc(-n3c4ccccc4c4cc(-c5ccc6c(c5)c5ccccc5n6-c5cc(-c6ccccc6)nc(-c6ccccc6)n5)ccc43)c2)cc1 ; c1ccc(-c2cc(-c3ccccc3)cc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cccc(-n3c4ccccc4c4cc(-c5ccc6c(c5)c5ccccc5n6-c5cc(-c6ccccc6)nc(-c6ccccc6)c5)ccc43)c2)cc1]

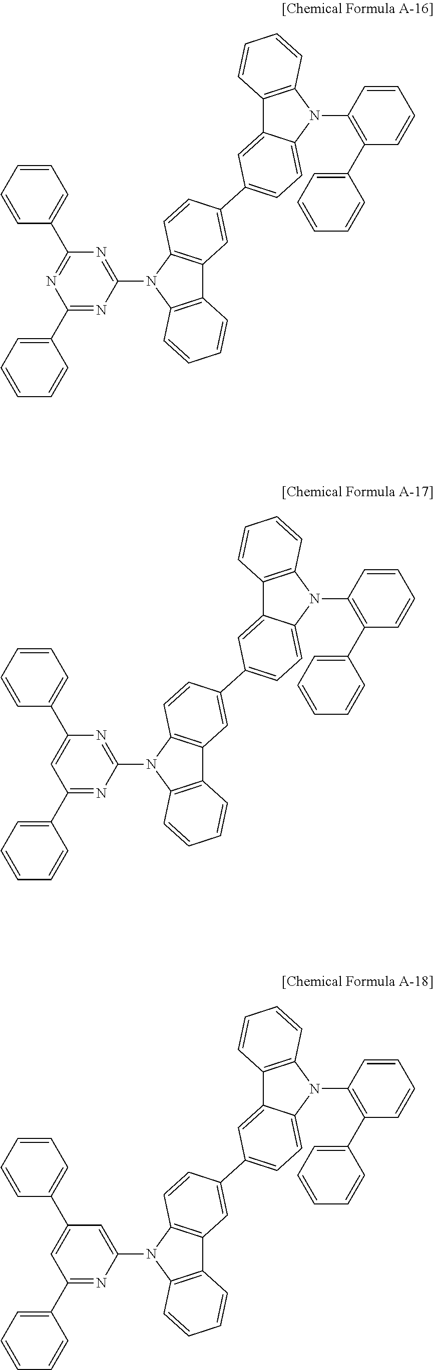 [SMILES: c1ccc(-c2nc(-c3ccccc3)nc(-n3c4ccccc4c4cc(-c5ccc6c(c5)c5ccccc5n6-c5ccccc5-c5ccccc5)ccc43)n2)cc1 ; c1ccc(-c2cc(-c3ccccc3)nc(-n3c4ccccc4c4cc(-c5ccc6c(c5)c5ccccc5n6-c5ccccc5-c5ccccc5)ccc43)n2)cc1 ; c1ccc(-c2cc(-c3ccccc3)nc(-n3c4ccccc4c4cc(-c5ccc6c(c5)c5ccccc5n6-c5ccccc5-c5ccccc5)ccc43)c2)cc1]

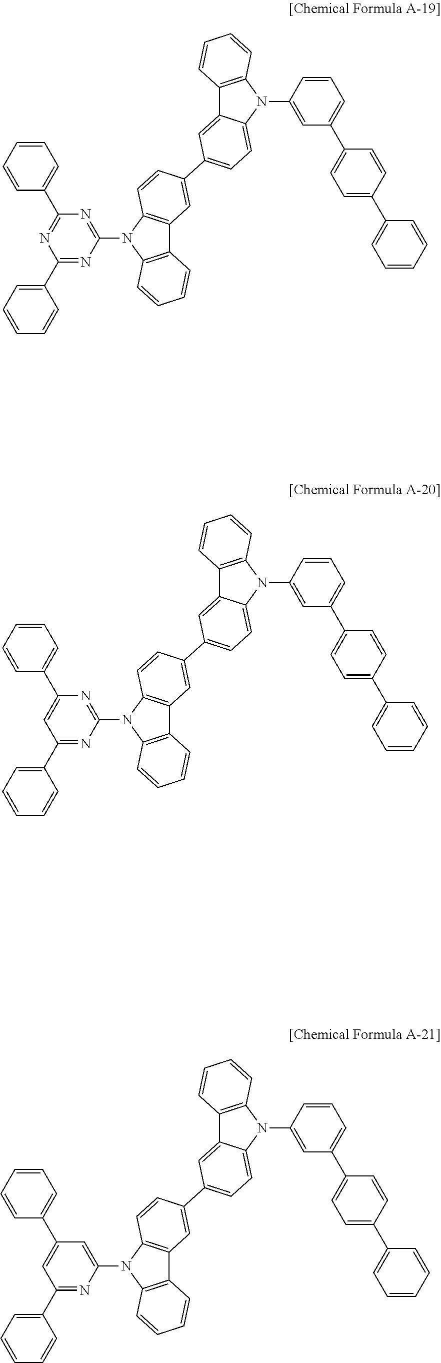 [SMILES: c1ccc(-c2ccc(-c3cccc(-n4c5ccccc5c5cc(-c6ccc7c(c6)c6ccccc6n7-c6nc(-c7ccccc7)nc(-c7ccccc7)n6)ccc54)c3)cc2)cc1 ; c1ccc(-c2ccc(-c3cccc(-n4c5ccccc5c5cc(-c6ccc7c(c6)c6ccccc6n7-c6nc(-c7ccccc7)cc(-c7ccccc7)n6)ccc54)c3)cc2)cc1 ; c1ccc(-c2ccc(-c3cccc(-n4c5ccccc5c5cc(-c6ccc7c(c6)c6ccccc6n7-c6cc(-c7ccccc7)cc(-c7ccccc7)n6)ccc54)c3)cc2)cc1]

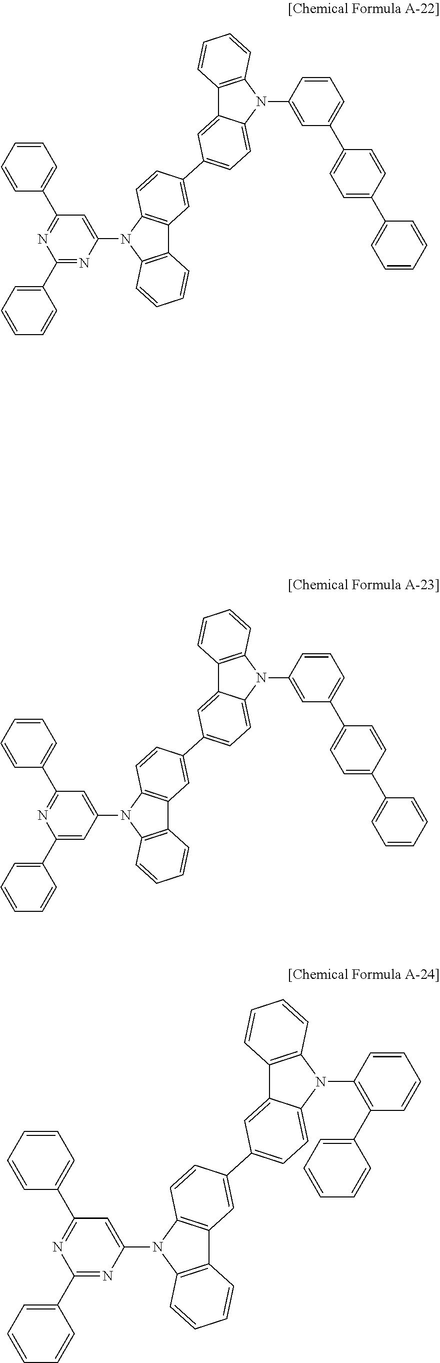 [SMILES: c1ccc(-c2ccc(-c3cccc(-n4c5ccccc5c5cc(-c6ccc7c(c6)c6ccccc6n7-c6cc(-c7ccccc7)nc(-c7ccccc7)n6)ccc54)c3)cc2)cc1 ; c1ccc(-c2ccc(-c3cccc(-n4c5ccccc5c5cc(-c6ccc7c(c6)c6ccccc6n7-c6cc(-c7ccccc7)nc(-c7ccccc7)c6)ccc54)c3)cc2)cc1 ; c1ccc(-c2cc(-n3c4ccccc4c4cc(-c5ccc6c(c5)c5ccccc5n6-c5ccccc5-c5ccccc5)ccc43)nc(-c3ccccc3)n2)cc1]

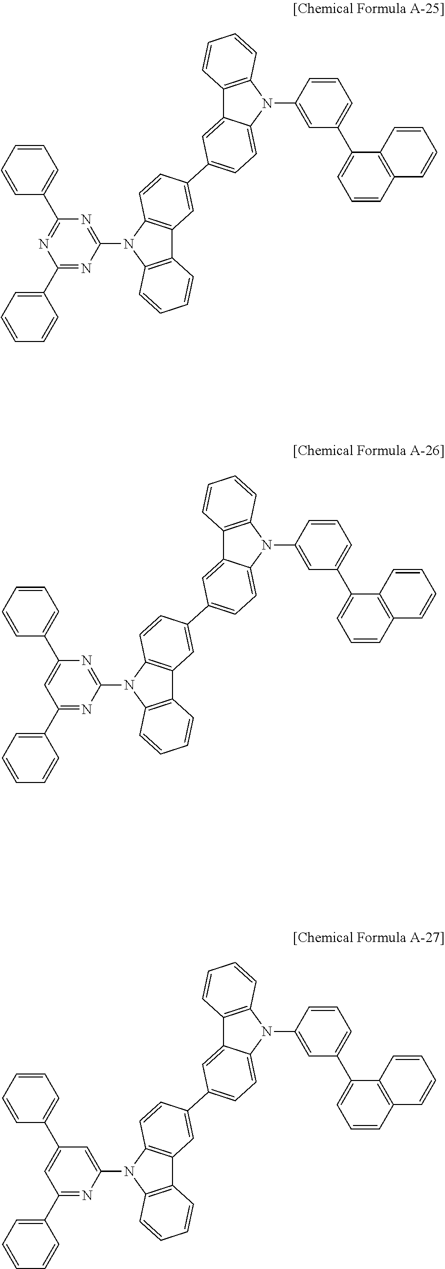 [SMILES: c1ccc(-c2nc(-c3ccccc3)nc(-n3c4ccccc4c4cc(-c5ccc6c(c5)c5ccccc5n6-c5cccc(-c6cccc7ccccc67)c5)ccc43)n2)cc1 ; c1ccc(-c2cc(-c3ccccc3)nc(-n3c4ccccc4c4cc(-c5ccc6c(c5)c5ccccc5n6-c5cccc(-c6cccc7ccccc67)c5)ccc43)n2)cc1 ; c1ccc(-c2cc(-c3ccccc3)nc(-n3c4ccccc4c4cc(-c5ccc6c(c5)c5ccccc5n6-c5cccc(-c6cccc7ccccc67)c5)ccc43)c2)cc1]

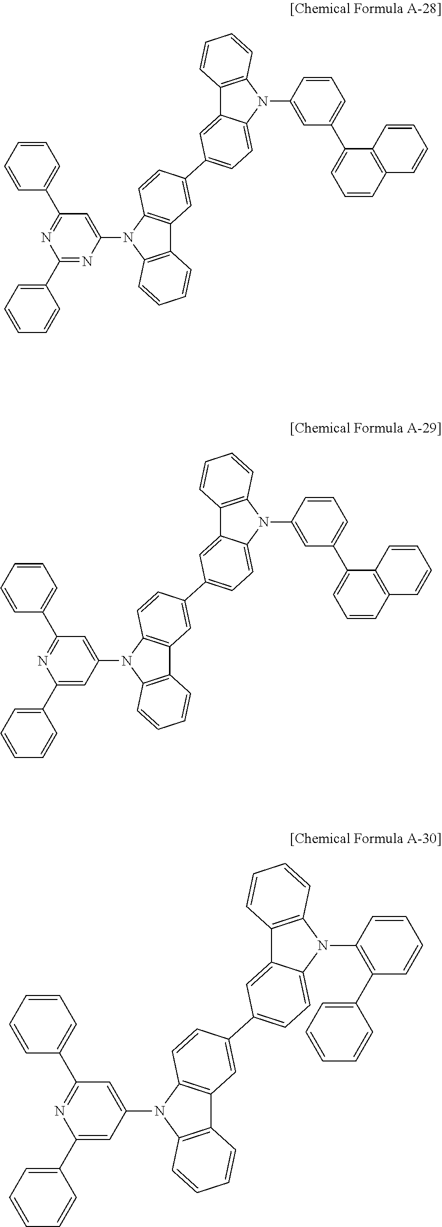 [SMILES: c1ccc(-c2cc(-n3c4ccccc4c4cc(-c5ccc6c(c5)c5ccccc5n6-c5cccc(-c6cccc7ccccc67)c5)ccc43)nc(-c3ccccc3)n2)cc1 ; c1ccc(-c2cc(-n3c4ccccc4c4cc(-c5ccc6c(c5)c5ccccc5n6-c5cccc(-c6cccc7ccccc67)c5)ccc43)cc(-c3ccccc3)n2)cc1 ; c1ccc(-c2cc(-n3c4ccccc4c4cc(-c5ccc6c(c5)c5ccccc5n6-c5ccccc5-c5ccccc5)ccc43)cc(-c3ccccc3)n2)cc1]

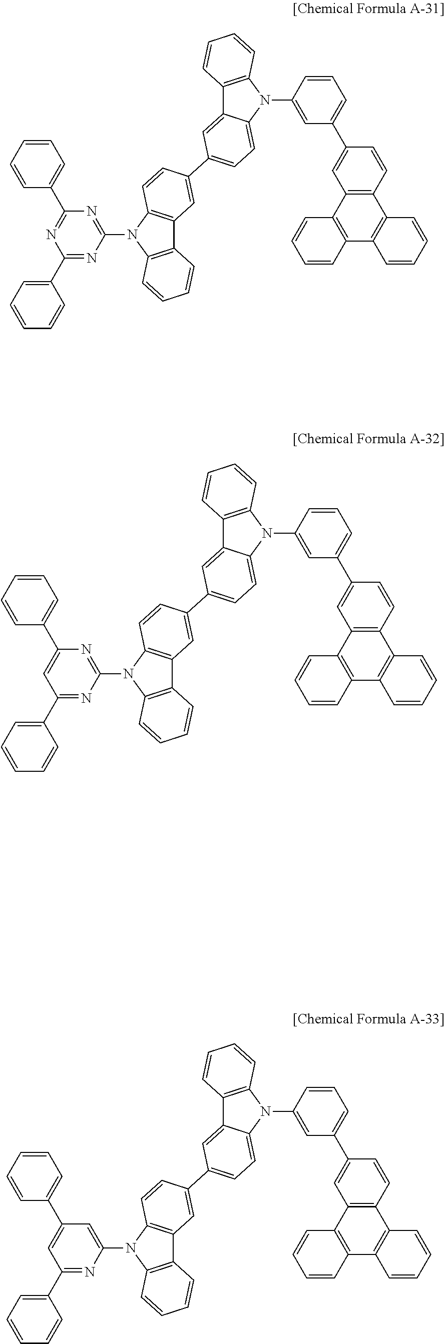 [SMILES: c1ccc(-c2nc(-c3ccccc3)nc(-n3c4ccccc4c4cc(-c5ccc6c(c5)c5ccccc5n6-c5cccc(-c6ccc7c8ccccc8c8ccccc8c7c6)c5)ccc43)n2)cc1 ; c1ccc(-c2cc(-c3ccccc3)nc(-n3c4ccccc4c4cc(-c5ccc6c(c5)c5ccccc5n6-c5cccc(-c6ccc7c8ccccc8c8ccccc8c7c6)c5)ccc43)n2)cc1 ; c1ccc(-c2cc(-c3ccccc3)nc(-n3c4ccccc4c4cc(-c5ccc6c(c5)c5ccccc5n6-c5cccc(-c6ccc7c8ccccc8c8ccccc8c7c6)c5)ccc43)c2)cc1]

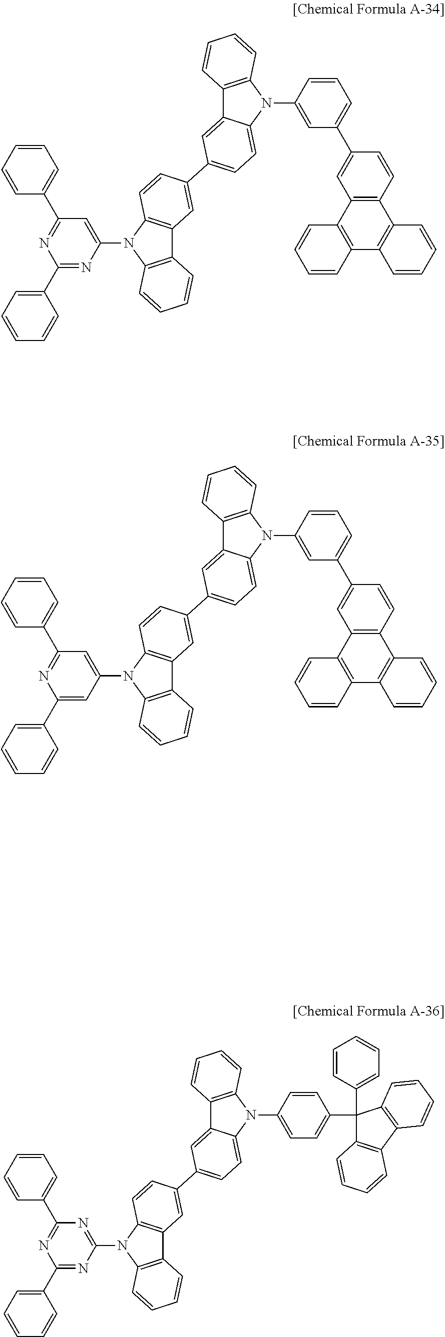 [SMILES: c1ccc(-c2cc(-n3c4ccccc4c4cc(-c5ccc6c(c5)c5ccccc5n6-c5cccc(-c6ccc7c8ccccc8c8ccccc8c7c6)c5)ccc43)nc(-c3ccccc3)n2)cc1 ; c1ccc(-c2cc(-n3c4ccccc4c4cc(-c5ccc6c(c5)c5ccccc5n6-c5cccc(-c6ccc7c8ccccc8c8ccccc8c7c6)c5)ccc43)cc(-c3ccccc3)n2)cc1 ; c1ccc(-c2nc(-c3ccccc3)nc(-n3c4ccccc4c4cc(-c5ccc6c(c5)c5ccccc5n6-c5ccc(C6(c7ccccc7)c7ccccc7-c7ccccc76)cc5)ccc43)n2)cc1]

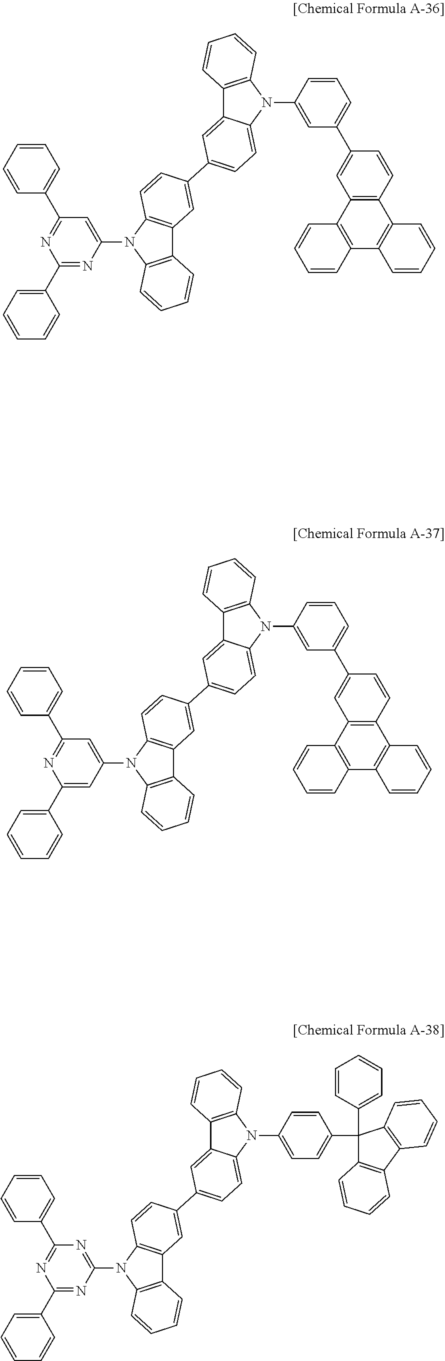 [SMILES: c1ccc(-c2cc(-n3c4ccccc4c4cc(-c5ccc6c(c5)c5ccccc5n6-c5cccc(-c6ccc7c8ccccc8c8ccccc8c7c6)c5)ccc43)nc(-c3ccccc3)n2)cc1 ; c1ccc(-c2cc(-n3c4ccccc4c4cc(-c5ccc6c(c5)c5ccccc5n6-c5cccc(-c6ccc7c8ccccc8c8ccccc8c7c6)c5)ccc43)cc(-c3ccccc3)n2)cc1 ; c1ccc(-c2nc(-c3ccccc3)nc(-n3c4ccccc4c4cc(-c5ccc6c(c5)c5ccccc5n6-c5ccc(C6(c7ccccc7)c7ccccc7-c7ccccc76)cc5)ccc43)n2)cc1]

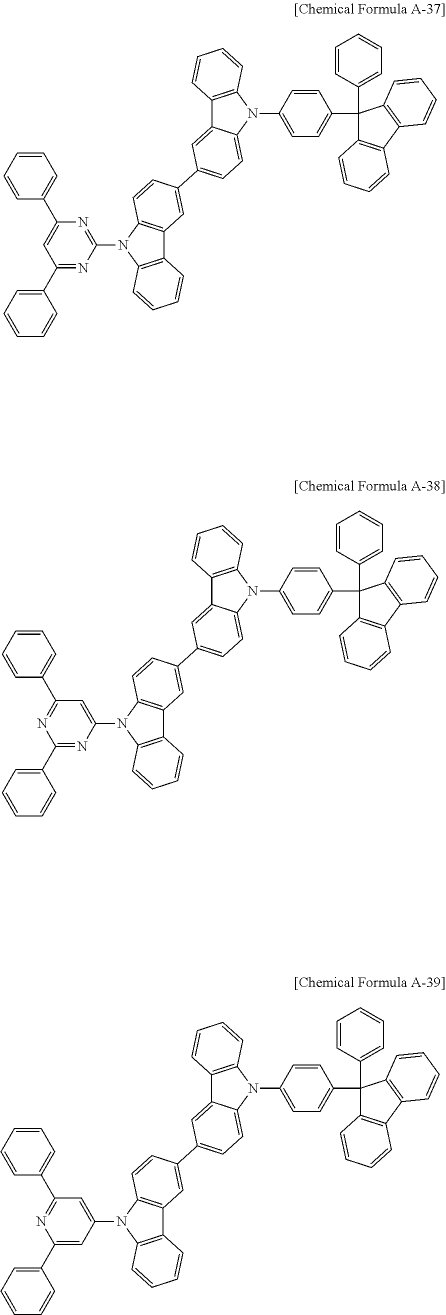 [SMILES: c1ccc(-c2cc(-c3ccccc3)nc(-n3c4ccccc4c4cc(-c5ccc6c(c5)c5ccccc5n6-c5ccc(C6(c7ccccc7)c7ccccc7-c7ccccc76)cc5)ccc43)n2)cc1 ; c1ccc(-c2cc(-n3c4ccccc4c4cc(-c5ccc6c(c5)c5ccccc5n6-c5ccc(C6(c7ccccc7)c7ccccc7-c7ccccc76)cc5)ccc43)nc(-c3ccccc3)n2)cc1 ; c1ccc(-c2cc(-n3c4ccccc4c4cc(-c5ccc6c(c5)c5ccccc5n6-c5ccc(C6(c7ccccc7)c7ccccc7-c7ccccc76)cc5)ccc43)cc(-c3ccccc3)n2)cc1]

8. The compound for an organic optoelectronic device as claimed in claim 1, wherein the compound for an organic optoelectronic device is represented by one of the following Chemical Formulae B-1 to B-25:

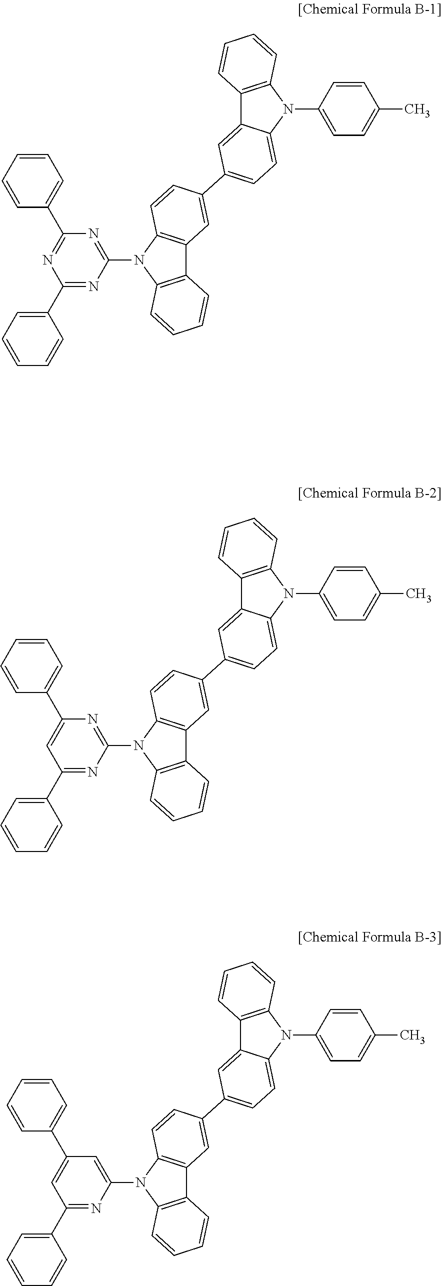 [SMILES: Cc1ccc(-n2c3ccccc3c3cc(-c4ccc5c(c4)c4ccccc4n5-c4nc(-c5ccccc5)nc(-c5ccccc5)n4)ccc32)cc1 ; Cc1ccc(-n2c3ccccc3c3cc(-c4ccc5c(c4)c4ccccc4n5-c4nc(-c5ccccc5)cc(-c5ccccc5)n4)ccc32)cc1 ; Cc1ccc(-n2c3ccccc3c3cc(-c4ccc5c(c4)c4ccccc4n5-c4cc(-c5ccccc5)cc(-c5ccccc5)n4)ccc32)cc1]

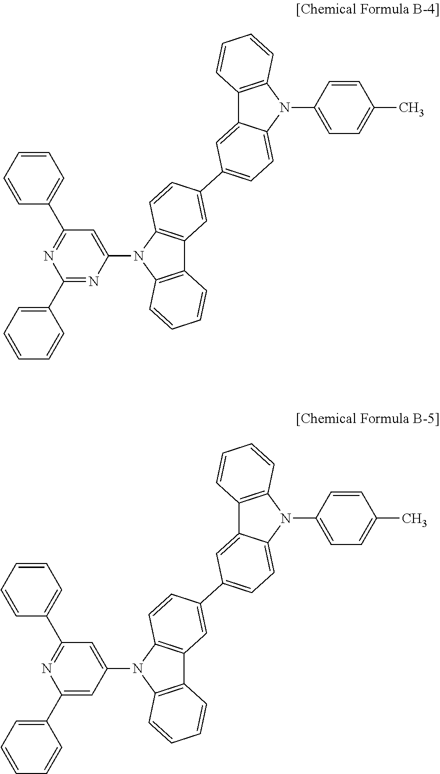 [SMILES: Cc1ccc(-n2c3ccccc3c3cc(-c4ccc5c(c4)c4ccccc4n5-c4cc(-c5ccccc5)nc(-c5ccccc5)n4)ccc32)cc1 ; Cc1ccc(-n2c3ccccc3c3cc(-c4ccc5c(c4)c4ccccc4n5-c4cc(-c5ccccc5)nc(-c5ccccc5)c4)ccc32)cc1]

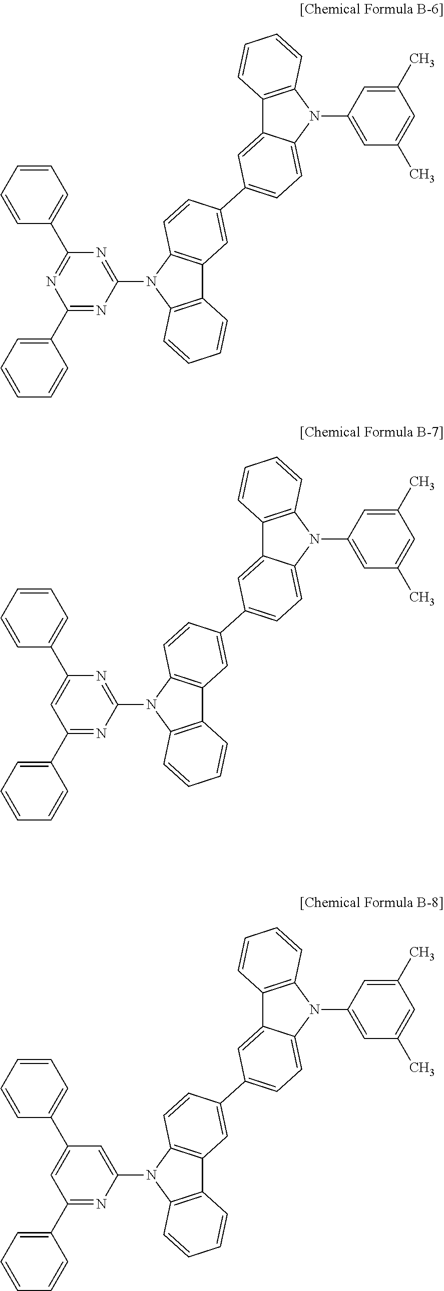 [SMILES: Cc1cc(C)cc(-n2c3ccccc3c3cc(-c4ccc5c(c4)c4ccccc4n5-c4nc(-c5ccccc5)nc(-c5ccccc5)n4)ccc32)c1 ; Cc1cc(C)cc(-n2c3ccccc3c3cc(-c4ccc5c(c4)c4ccccc4n5-c4nc(-c5ccccc5)cc(-c5ccccc5)n4)ccc32)c1 ; Cc1cc(C)cc(-n2c3ccccc3c3cc(-c4ccc5c(c4)c4ccccc4n5-c4cc(-c5ccccc5)cc(-c5ccccc5)n4)ccc32)c1]

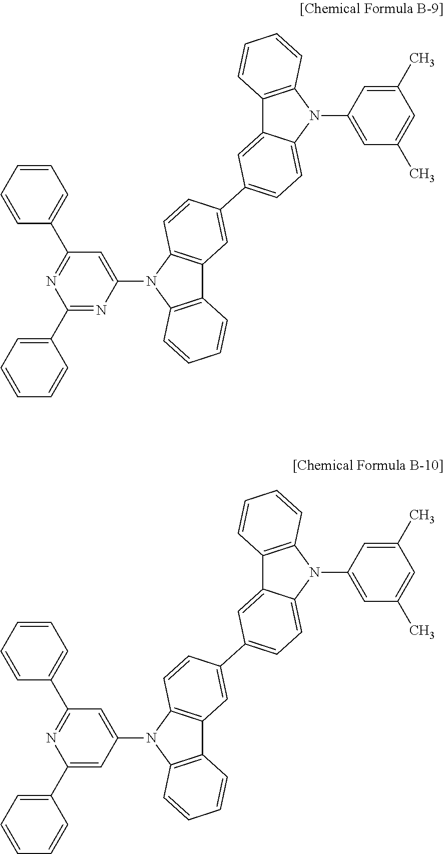 [SMILES: Cc1cc(C)cc(-n2c3ccccc3c3cc(-c4ccc5c(c4)c4ccccc4n5-c4cc(-c5ccccc5)nc(-c5ccccc5)n4)ccc32)c1 ; Cc1cc(C)cc(-n2c3ccccc3c3cc(-c4ccc5c(c4)c4ccccc4n5-c4cc(-c5ccccc5)nc(-c5ccccc5)c4)ccc32)c1]

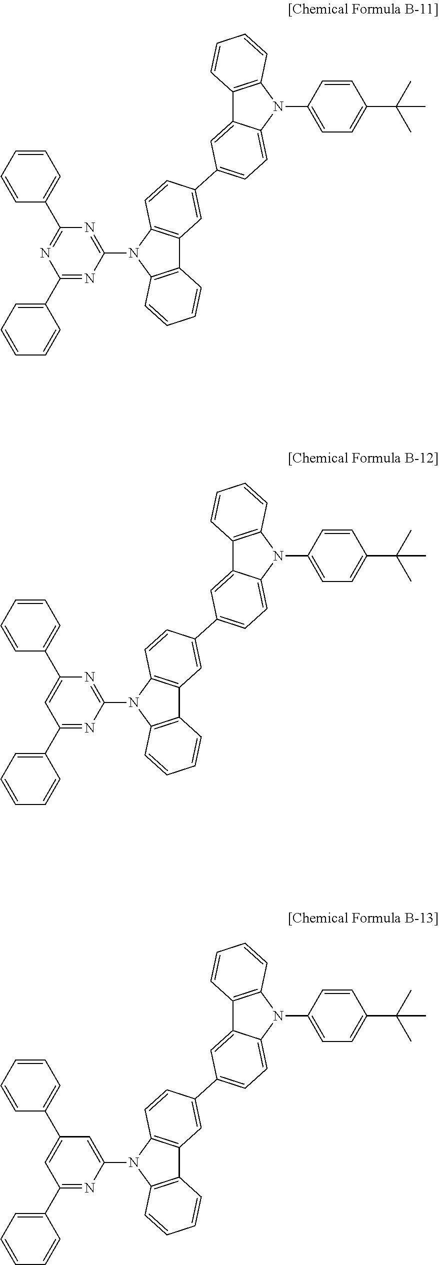 [SMILES: CC(C)(C)c1ccc(-n2c3ccccc3c3cc(-c4ccc5c(c4)c4ccccc4n5-c4nc(-c5ccccc5)nc(-c5ccccc5)n4)ccc32)cc1 ; CC(C)(C)c1ccc(-n2c3ccccc3c3cc(-c4ccc5c(c4)c4ccccc4n5-c4nc(-c5ccccc5)cc(-c5ccccc5)n4)ccc32)cc1 ; CC(C)(C)c1ccc(-n2c3ccccc3c3cc(-c4ccc5c(c4)c4ccccc4n5-c4cc(-c5ccccc5)cc(-c5ccccc5)n4)ccc32)cc1]

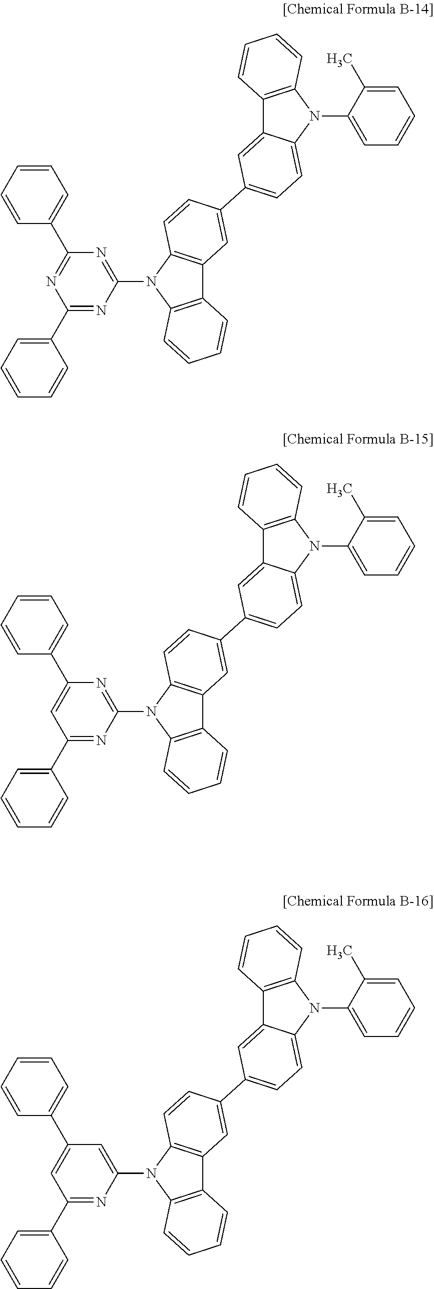 [SMILES: Cc1ccccc1-n1c2ccccc2c2cc(-c3ccc4c(c3)c3ccccc3n4-c3nc(-c4ccccc4)nc(-c4ccccc4)n3)ccc21 ; Cc1ccccc1-n1c2ccccc2c2cc(-c3ccc4c(c3)c3ccccc3n4-c3nc(-c4ccccc4)cc(-c4ccccc4)n3)ccc21 ; Cc1ccccc1-n1c2ccccc2c2cc(-c3ccc4c(c3)c3ccccc3n4-c3cc(-c4ccccc4)cc(-c4ccccc4)n3)ccc21]

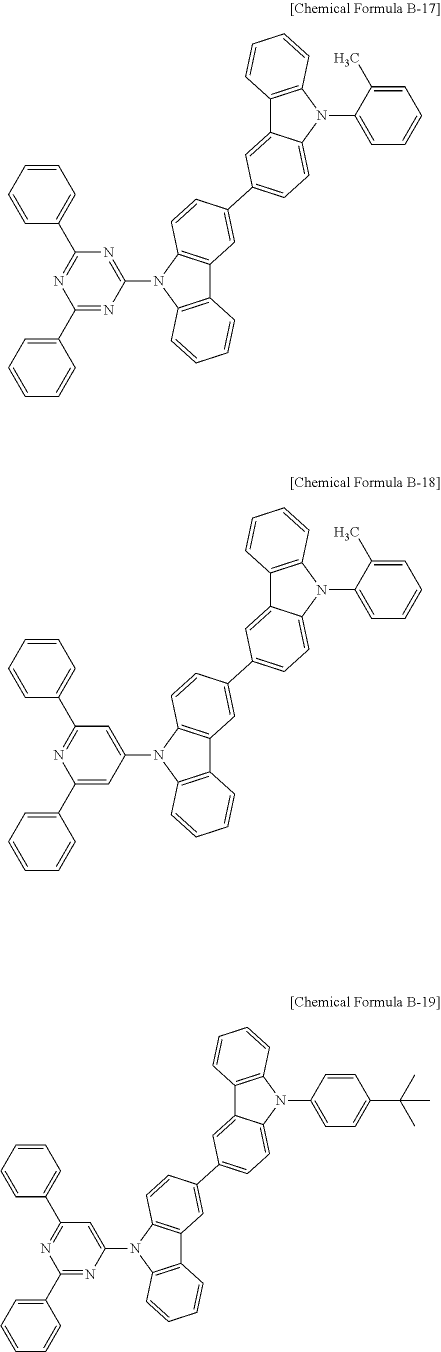 [SMILES: Cc1ccccc1-n1c2ccccc2c2cc(-c3ccc4c(c3)c3ccccc3n4-c3nc(-c4ccccc4)nc(-c4ccccc4)n3)ccc21 ; Cc1ccccc1-n1c2ccccc2c2cc(-c3ccc4c(c3)c3ccccc3n4-c3cc(-c4ccccc4)nc(-c4ccccc4)c3)ccc21 ; CC(C)(C)c1ccc(-n2c3ccccc3c3cc(-c4ccc5c(c4)c4ccccc4n5-c4cc(-c5ccccc5)nc(-c5ccccc5)n4)ccc32)cc1]

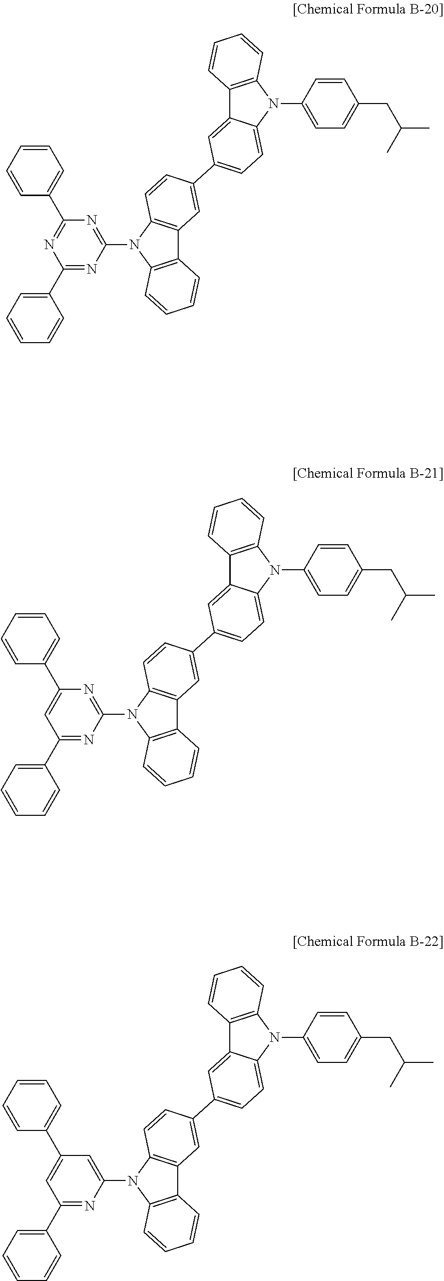 [SMILES: CC(C)Cc1ccc(-n2c3ccccc3c3cc(-c4ccc5c(c4)c4ccccc4n5-c4nc(-c5ccccc5)nc(-c5ccccc5)n4)ccc32)cc1 ; CC(C)Cc1ccc(-n2c3ccccc3c3cc(-c4ccc5c(c4)c4ccccc4n5-c4nc(-c5ccccc5)cc(-c5ccccc5)n4)ccc32)cc1 ; CC(C)Cc1ccc(-n2c3ccccc3c3cc(-c4ccc5c(c4)c4ccccc4n5-c4cc(-c5ccccc5)cc(-c5ccccc5)n4)ccc32)cc1]

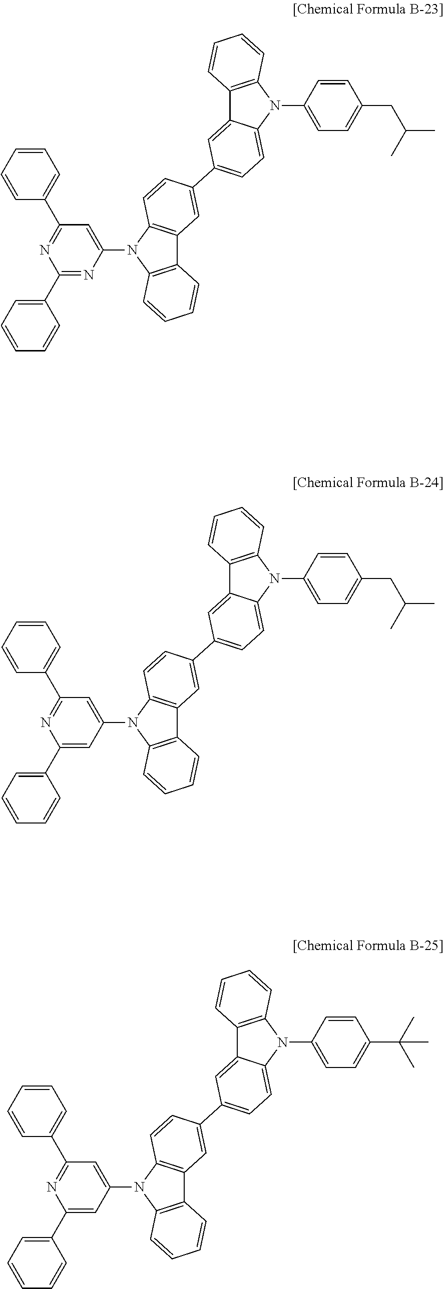 [SMILES: CC(C)Cc1ccc(-n2c3ccccc3c3cc(-c4ccc5c(c4)c4ccccc4n5-c4cc(-c5ccccc5)nc(-c5ccccc5)n4)ccc32)cc1 ; CC(C)Cc1ccc(-n2c3ccccc3c3cc(-c4ccc5c(c4)c4ccccc4n5-c4cc(-c5ccccc5)nc(-c5ccccc5)c4)ccc32)cc1 ; CC(C)(C)c1ccc(-n2c3ccccc3c3cc(-c4ccc5c(c4)c4ccccc4n5-c4cc(-c5ccccc5)nc(-c5ccccc5)c4)ccc32)cc1]

9. The compound for an organic optoelectronic device as claimed in claim 1, wherein the compound for an organic optoelectronic device has a triplet excitation energy (T1) of 2.0 eV or more.

10. The compound for an organic optoelectronic device as claimed in claim 1, wherein the organic optoelectronic device is selected from an organic photoelectric device, an organic light emitting diode, an organic solar cell, an organic transistor, an organic photo-conductor drum, and an organic memory device.

11. An organic light emitting diode, comprising:
an anode,
a cathode, and
at least one organic thin layer between the anode and the cathode,
wherein the at least one organic thin layer includes the compound for an organic optoelectronic device as claimed in claim 1.

12. The organic light emitting diode as claimed in claim 11, wherein the at least one organic thin layer is selected from an emission layer, a hole transport layer (HTL), a hole injection layer (HIL), an electron transport layer (ETL), an electron injection layer (EIL), a hole blocking layer, and a combination thereof.

13. The organic light emitting diode as claimed in claim 12, wherein:
the at least one organic thin layer includes the hole transport layer (HTL) or the hole injection layer (HIL), and
the compound for an organic optoelectronic device is included in the hole transport layer (HTL) or the hole injection layer (HIL).

14. The organic light emitting diode as claimed in claim 12, wherein:
the at least one organic thin layer includes the emission layer, and
the compound for an organic optoelectronic device is included in the emission layer.

15. The organic light emitting diode as claimed in claim 14, wherein:
the at least one organic thin layer includes the emission layer, and
the compound for an organic optoelectronic device is a phosphorescent or fluorescent host material in the emission layer.

16. A display device including the organic light emitting diode as claimed in claim 11.
